(19) United States
(12) Patent Application Publication
(10) Pub. No.: US 2016/0175829 A1
(43) Pub. Date: Jun. 23, 2016

(54) CATALYST SYSTEMS FOR USE IN CONTINUOUS FLOW REACTORS AND METHODS OF MANUFACTURE AND USE THEREOF

(71) Applicant: THE GOVERNORS OF THE UNIVERSITY OF ALBERTA, Edmonton (CA)

(72) Inventors: Steven H. BERGENS, Edmonton (CA); Suneth KALAPUGAMA, Edmonton (CA); Prabin NEPAL, Edmonton (CA); Elizabeth MCGINITIE, Edmonton (CA)

(73) Assignee: THE GOVERNORS OF THE UNIVERSITY OF ALBERTA, Edmonton, AB (CA)

(21) Appl. No.: 14/909,683

(22) PCT Filed: Aug. 4, 2014
(86) PCT No.: PCT/CA2014/050732

§ 371 Date: Feb. 2, 2016

Related U.S. Application Data
(60) Provisional application No. 61/861,656, filed on Aug. 2, 2013.

(51) Int. Cl. B01J 31/24; B01J 19/24; C07C 29/56; C07B 35/02; C07C 45/00; C07C 231/12; C07C 51/36; C07C 67/303; B01J 37/02; C07F 15/00

(52) U.S. Cl. CPC B01J 31/2452; B01J 37/0203; B01J 19/24; C07C 29/56; C07F 15/0073; C07C 45/00; C07C 231/12; C07C 51/36; C07C 67/303; C07B 35/02; B01J 2231/52; B01J 2231/645; B01J 2531/822; B01J 2531/824; B01J 2531/821; B01J 2219/24

(57) ABSTRACT

The present application provides a composite material and system for use in a heterogeneous flow reactor, which can include: a catalytic polymeric framework comprising catalyst-containing monomeric units each separated by at least one non-catalyst-containing monomeric unit; and a solid support material, wherein the catalytic polymeric framework is covalently or non-covalently immobilized on or in the support material. Each catalyst-containing monomeric subunit in the polymeric framework comprises a transition metal bound to a catalyst ligand. Also provided are methods of manufacture and use of the catalyst system and composite material.

### FIELD OF THE INVENTION

The present application pertains to the field of asymmetric catalysis. More particularly, the present application relates to a heterogeneous system and method for asymmetric catalysis.

### INTRODUCTION

Asymmetric catalysis is enantioselective conversion of a prochiral substrate into a chiral product in the presence of a chiral homogeneous catalyst. Asymmetric catalysis offers exceptional versatility; chiral homogeneous catalysts can be readily tailored and/or modified for any desired reaction. Additionally, use of catalysts in synthesis is generally considered to be more environmentally friendly than use of stoichiometric reagents. Asymmetric catalysis is used in industrial synthesis of a variety of natural products. One such example is the rhodium-(S)-BINAP ((S)-BINAP=(S)-2,2′-bis(diphenylphosphino)-1,1′-binaphthyl)) catalyzed isomerization of N,N-diethylgeranylamine to give, after hydrolysis, enantiopure (R)-citronellal, developed by Ryoji Noyori, recipient of the 2001 Nobel Prize in Chemistry [(Tani, K.; Yamagata, T.; Otsuka, S.; Akutagawa, S.; Kumobayashi, H.; Taketomi, T.; Takaya, H.; Miyashita, A.; Noyori, R. J. Am. Chem. Soc., Chem. Commun. 1982, 600; Tani, K.; Yamagata, T.; Akutagawa, S.; Kumobayashi, H.; Taketomi, T.; Takaya, H.; Miyashita, A.; Noyori, R.; Otsuka, S. J. Am. Chem. Soc. 1984, 106, 5208; Inoue, S.-I.; Takaya, H.; Tani, K.; Otsuka, S.; Sato, T.; Noyori, R. J. Am. Chem. Soc. 1990, 112, 4897]. This reaction is a key step in industrial synthesis of (−)-menthol, a common aesthetic.

Despite advantages of asymmetric catalysis, there are inherent challenges that affect its utility and applicability. Homogeneous catalysts can be toxic due to the presence of transition metal centers, which is a serious concern for pharmaceutical industries [Garrett, C. E.; Prasad, K. Adv. Synth. Catal. 2004, 346, 889]. This can result in costly and time-consuming work-ups to separate catalytic residues from desired product(s). Homogeneous catalysts are also known to decompose during work-up, preventing catalyst recycling. They are also often air sensitive and expensive; chiral ligands can be more costly than transition metal precursor themselves [Hawkins, J. M.; Watson, T. J. N. Angew. Chem. Int. Ed. 2004, 43, 3224].

Consequently, research has been directed toward immobilization of chiral catalysts in an effort to reduce costs, and provide more sustainable industrial processes for production of enantiopure compounds [Asymmetric Catalysis on Industrial Scale; Blaser, H. U., Schmidt, E., Eds.; Wiley-VHC: Weinheim, Germany, 2003; Chiral Catalyst Immobilization and Recycling; De Vos, D. E., Vankelecom, I. F. J., Jacobs, P. A., Eds.; Wiley-VHC: Weinheim, Germany, 2008]. Successful immobilization of homogeneous catalysts may offer easy catalyst isolation from product mixtures, increased potential for recyclability, high catalytic efficiencies, and rapid screening of potential ligand sets. Immobilized homogeneous catalysts may also function quite effectively in continuous flow processes, potentially increasing chiral compound production while reducing catalyst cost, heavy metal contamination, and product decomposition [Kirschning, A.; Jas, G. Immobilized Catalysts Topic in Current Chemistry 2004, 242, 209; Nagy, K. D. (2012). Catalyst Immobilization Techniques for Continuous Flow Synthesis. Ph. D. Thesis. Massachusetts Institute of Technology: Cambridge; Chen, B.; Dingerdissen, U.; Krauter, J. G. E.; Rotgerink. H.; Mobus, K.; Ostgard, D. J.; Panster, P.; Riermeier, T. H.; Seebald, S.; Tacke, T.; Trauthwein, H. Appl. Catal. A: General 2005, 280, 17; Balogh at al. Green Chem. 2012, 14, 1146; Shi et al. Chem. Eur. J. 2009, 15, 9855-9867].

Various approaches have been developed for immobilization of homogeneous catalysts, of which two more general methods involve non-covalent [Fraile, J. M.; Garcia, J. I.; Mayoral, J. A. Chem. Rev. 2009, 109, 360; Heitbaum, M.; Glorius, F.; Escher, I. Angew. Chem. Int. Ed. 2006, 45, 4732.; McMorn, P.; Hutchings, G.; Chem. Soc. Rev. 2004, 33, 108; Zhao, X. S.; Bao, X. Y.; Guo, W.; Lee, F. Y. Mater. Today 2006, 9, 32] and covalent interactions [Dioos, B. M. L.; Vankelecom, I. F. J.; Jacobs, P. A. Adv. Synth. Catal. 2006, 348, 1413; Leadbeater, N. E.; Marco, M. Chem. Rev. 2002, 102, 3217; Fan, Q-H.; Li, Y.-M.; Chan, A. S. C. Chem. Rev. 2002, 102, 3385; Wang, Z.; Chen, G.; Ding, K. Chem. Rev. 2009, 109, 322; Ding, K.; Wang, Z.; Wang, X.; Liang, Y.; Wang, X. Chem.-Eur. J. 2006, 12, 5188] between a metal center and support, or between a chiral ligand and support. Non-covalent methods of immobilization include electrostatic interactions between ionic catalysts and supports, adsorption of a catalyst onto a support, and entrapment of a catalyst within a support (FIG. 1). Covalent methods of immobilization include formation of a direct metal-support bond, or formation of a direct modified ligand-support bond (FIG. 2).

Despite recent advances, non-covalently immobilized catalysts continue to have poor activity as compared to their homogenous analogues, and attempts at catalyst recycling have been challenging (<3 cycles) [Chiral Catalyst Immobilization and Recycling; De Vos, D. E., Vankelecom, I. F. J., Jacobs, P. A., Eds.; Wiley-VHC: Weinheim, Germany, 2008]. Significant metal leaching can occur over a catalyst's lifetime due to a relatively weak interaction between the catalyst and support, resulting in poor activity and reusability. Consequently, research has focused on covalent immobilization as a means of preventing significant metal leaching and loss of catalytic activity. Covalently immobilized catalysts, however, can suffer from unpredictable activities and selectivities due to changes in electronic environment of their metal center(s) upon formation of direct metal-support, or ligand-support bonds. As a result, polymer-supported asymmetric catalysts have been developed, either by copolymerization of modified catalyst ligands, or grafting modified ligands onto polymeric supports. Polymerization as an immobilization method can provide good catalyst-support interactions, while limiting metal leaching and increasing reusability. Provided that polymerized units and/or polymerizable functional groups are incorporated into a catalyst's ligands, it can also offer a significant degree of synthetic control, and can potentially limit support effects on a metal center's electronic environment.

Polymer-supported immobilized catalysts have been synthesized via grafting onto polymeric resins [Bayston, D. J.; Fraser, J. L.; Ashton, M. R.; Baxter, A. D.; Polywka, M. E. C.; Moses, E. J. Org. Chem. 1998, 63, 3137; Chapuis, C.; Barthe, M.; de Saint Laumer, J.-Y.; Helv. Chim. Acta 2001, 84, 230; Song, C. E.; Yang, J. W.; Roh, E. J.; Lee, S.-G.; Ahn, J. H.; Han, H. Angew. Chem. Int. Ed. 2002, 41, 3852.], radical copolymerization of vinyl derivatives of arenes and phosphines [Bianchini, C.; Frediani, M.; Mantovani, G.; Vizza, F. Organometallics 2001, 20, 2660; Bianchini, C.; Frediani, M.; Vizza, F. Chem. Commun. 2001, 479; Deschenaux, R.; Stille, J. K. J. Org. Chem. 1985, 50, 2299], condensation reactions between acid derivatives and amines or alcohols [Deng, G. J.; Fan, Q. H.; Chen, X. M.; Liu, D. S.; Chan, A. S. C. Chem. Commun. 2002, 1570; Fan, Q. H.; Ren, C. Y.; Yeung, C. H.; Hu, W. H.; Chan, A. S. C. J. Am. Chem. Soc. 1999, 121, 7407], condensation polymerizations between amines and isocyanates [Saluzzo, C.; Lamouille, T.; Herault, D.; Lemaaire, M. Bioorg. Med. Chem. Lett. 2002, 12, 1841; Saluzzo, C.; ter Halle, R.; Touchard, F.; Fache, F.; Schulz, E.; Leamire, M. J. Organomet. Chem. 2000, 603, 30; ter Halle, R.; Colasson, B.; Schulz, E.; Spagnol, M.; Lemaire, M. Tetrahedron Lett. 2000, 41, 643; ter Halle, R.; Schulz, E.; Spagnol, M.; Lemaire, M. Synlett 2000, 680], and Suzuki-type couplings [Pu, L. Chem. Rev. 1998, 98, 2405. (b) Pu, L. Chem. Eur. J. 1999, 5, 2227. (c) Yu, H. B.; Hu, Q. S.; Pu, L. J. Am. Chem. Soc. 2000, 122, 6500]. Given that metal centers can interfere with these reactions, metallation of a system usually occurs after polymerization [Buchmeiser, M. R.; Kroll, R.; Wurst, K.; Schareina, T.; Kempe, R.; Eschbaumer, C.; Schubert, U. S. Macromol. Symp. 2001, 164 (Reactive Polymers), 187]. However, metallation may not be quantitative due to restricted access to some chelating ligand sites in a polymer's matrix; this may result in low catalyst loadings and wasted ligand [Pugin, B.; Blaser, H.-U. Top. Catal. 2010, 53, 953]. Additionally, an inherent lack of control over polymerization procesess can generate ill-defined polymeric systems with limited access to active sites. These factors can lead to poor catalyst performance for heterogenized systems as compared to their homogeneous analogues.

To address some of these limitations, Ru-BINAP and Rh-BINAP polymeric catalyst frameworks were developed (BINAP=2,2′-bis(diphenyl phosphino)-1,1′-binaphthyl) (FIGS. 3 and 4) [Ralph, C. K.; Akotsi, O. M.; Bergens, S. H. Organometallics 2004, 23, 1484; Ralph, C. K.; Bergens, S. H. Organometallics 2007, 26, 1571; Bergens, S. H.; Sullivan, A. D.; Hass, M. Heterogeneous Rhodium Metal Catalysts. 2010]. These frameworks were synthesized by directly polymerizing a metal-containing monomer (Ru-BINAP and Rh-BINAP, wherein the BINAP ligand was modified to incorporate polymerizable norbornene units) in the presence of a spacer monomer (e.g. cis-cyclooctene, COE) via alternating ring-opening metathesis polymerization (ROMP) [Ralph, C. K.; Bergens, S. H. Organometallics 2007, 26, 1571; Bergens, S. H.; Sullivan, A. D.; Hass, M. Heterogeneous Rhodium Metal Catalysts. 2010]. The resulting polymeric catalyst frameworks reportedly offered a high density of active catalytic sites within the polymer matrix.

The above information is provided for the purpose of making known information believed by the applicant to be of possible relevance to the present invention. No admission is necessarily intended, nor should be construed, that any of the preceding information constitutes prior art against the present invention.

### SUMMARY OF THE INVENTION

An object of the present application is to provide catalyst systems for use in heterogeneous reactors, such as flow reactors, and methods of manufacture and use thereof.

In accordance with an aspect of the application, there is provided a system for use in a heterogeneous flow reactor, comprising: a flow reactor cartridge containing a polymer-supported catalyst immobilized on and/or in a solid support material, wherein the polymer-supported catalyst comprises catalyst-containing monomer subunits incorporated in a polymer framework and wherein each catalyst-containing monomer subunit comprises a transition metal covalently bound to a catalyst ligand.

In accordance with another aspect of the application, there is provided a composite material comprising: (i) a catalytic polymeric framework comprising catalyst-containing monomeric units each separated by at least one non-catalyst-containing monomeric unit; and (ii) a solid support material, wherein the catalytic polymeric framework is covalently or non-covalently immobilized on and/or in said support material.

In one embodiment, the catalytic polymeric framework is derived from a transition metal catalyst, wherein the transition metal can be, for example, Cr, Mo, Fe, Ru, Os, Co, Rh, Ir, Ni, Pd, Pt, Cu, Ag, and/or Au.

In one embodiment the solid support material comprises BaSO₄, barium (L)- and (D)-tartrates, aluminum oxide (Al₂O₃), silica (SiO₂), Fe₃O₄, Teflon™, Celite™, AgCl, sand or any combination thereof.

In another embodiment, each catalyst-containing monomeric unit is derived from a monomer having the structure:

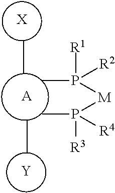

wherein

A is a substituted or unsubstituted aliphatic or aryl group;

X and Y are each independently a polymerizable moiety, wherein one of X or Y may be absent;

R¹, R², R³ and R⁴ are independently selected from aryl (e.g., phenyl), and C₄₋₈cycloalkyl, the latter two groups being unsubstituted or substituted, where possible, with 1, 2, 3, 4, or 5 groups independently selected from C₁₋₆alkyl, OC₁₋₆alkyl and halo, or R¹ and R² and/or R³ and R⁴ together with the atoms to which they are attached form a substituted or unsubstituted cycloalkyl; and

M is a transition metal, optionally bound to another ligand or combination of ligands.

In a more specific embodiment, the polymerizable moiety is selected from the group

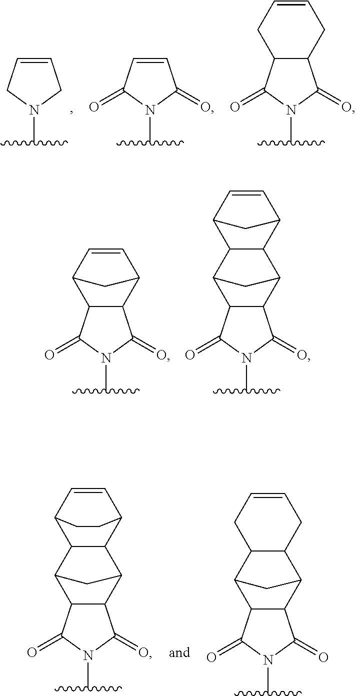

consisting of:

In an alternative embodiment, the composite material comprises a catalyst-containing monomer subunit that comprises

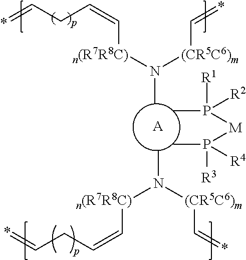

wherein
R¹, R², R³ and R⁴ are independently selected from phenyl and C₄₋₈cycloalkyl, the latter two groups being unsubstituted or substituted, where possible, with 1, 2, 3, 4, or 5 groups independently selected from C₁₋₆alkyl, OC₁₋₆alkyl and halo;

A is a binaphthyl group or a derivative of a binaphthyl group, each being unsubstituted or substituted with one or more groups independently selected from C₁₋₆alkyl, OC₁₋₆alkyl and halo;
R⁵, R⁶, R⁷ and R⁸ are independently selected from H, C₁₋₆alkyl, OC₁₋₆alkyl and halo; or R⁵ and R⁶ and/or R⁷ and R⁸ are ═O; or one of R⁵ and R⁶ is linked to one of R⁷ and R⁸ to form, together with the atoms to which they are attached and the atoms connecting them, a monocyclic, bicyclic or tricylic ring system; R⁵, R⁶, R⁷ and R⁸ in each methylene unit is the same or different, and means the double bond attached to this bond is in the cis or trans configuration, if applicable;
m and n are, independently, an integer between and including 0 and 10;
p is an integer between and including 1 and 14; and
M is the transition metal, optionally bound to another ligand or combination of ligands.

In another aspect of the application, there is provided a method for metal-catalyzed organic synthesis comprising flowing a substrate for an organic synthesis through a flow reactor system comprising the catalytic composite material described herein; and, optionally, isolating one or more products of the organic synthesis from the flow reactor system.

In accordance with another aspect of the present application, there is provided a method of preparing the catalytic composite material comprising a polymeric catalyst framework, said method comprising the steps of: (a) derivatizing a catalyst to add one or more polymerizable moieties to a ligand of the catalyst to form a catalyst-containing monomer; (b) polymerizing the catalyst-containing monomer with a non-catalyst-containing monomer using alternating ring-opening metathesis polymerization (ROMP) to form the catalytic polymeric framework; and (c) contacting the catalytic polymeric framework with a solid support material under conditions suitable for immobilization of the catalytic polymeric framework on and/or in the support material, via covalent or non-covalent interactions.

In accordance with another aspect of the present application, there is provided method of preparing a polymeric catalyst framework, said method comprising the steps of: (a) derivatizing a catalyst to add one or more polymerizable moieties to a ligand of the catalyst to form a catalyst-containing monomer; (b) polymerizing the catalyst-containing monomer with a non-catalyst-containing monomer using alternating ring-opening metathesis polymerization (ROMP) to form the catalytic polymeric framework, wherein the catalyst-containing monomer does not comprise a BINAP ligand, or wherein the polymerizable moiety does not comprise a norbornene. Also, provided by the present application are the polymeric catalyst frameworks prepared by this method.

In accordance with another aspect of the present application, there is provided a catalyst-containing monomer having the structure:

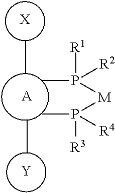

wherein

A is a substituted or unsubstituted aliphatic or aryl group;

X and Y are each independently a polymerizable moiety, wherein one of X or Y may be absent;

R¹, R², R³ and R⁴ are independently selected from aryl (e.g., phenyl), and C₄₋₈ cycloalkyl, the latter two groups being unsubstituted or substituted, where possible, with 1, 2, 3, 4, or 5 groups independently selected from C₁₋₆alkyl, OC₁₋₆alkyl and halo, or R¹ and R² and/or R³ and R⁴ together with the atoms to which they are attached form a substituted or unsubstituted cycloalkyl; and M is a transition metal (such as Cr, Mo, Fe, Ru, Os, Co, Rh, Ir, Ni, Pd, Pt, Cu, Ag, and/or Au), optionally bound to another ligand or combination of ligands, wherein the catalyst-containing monomer does not comprise a BINAP ligand, or wherein the polymerizable moiety does not comprise a norbornene.

### BRIEF DESCRIPTION OF THE FIGURES

For a better understanding of the present invention, as well as other aspects and further features thereof, reference is made to the following description which is to be used in conjunction with the accompanying drawings, where:

FIG. 1 schematically depicts non-covalent methods of immobilization of a catalyst on a support material;

FIG. 2 schematically depicts covalent methods of immobilization of a catalyst on a support material;

FIG. 3 schematically depicts a Ru-BINAP polymer-supported catalyst;

FIG. 4 schematically depicts a Rh-BINAP polymer-supported catalyst;

FIG. 5 depicts a schematic of an H-Cube®;

FIG. 6 schematically depicts a proposed mechanism of hydrogenation and isomerization via metal hydride intermediates;

FIG. 7 shows the ¹H NMR spectrum of [Pd((R,R)—NORPHOS)(η³-C₃H₅)]BF₄.

FIG. 8 shows the ¹H NMR spectrum of (S)-Phanephos oxide;

FIG. 9 shows the ³¹P {¹H}NMR spectrum of (S)-Phanephos oxide;

FIG. 10 shows the ³¹P {¹H}NMR spectrum of the product of (S)-Phanephos oxide nitration (crude);

FIG. 11 shows the ³¹P {(H}NMR spectrum of (S)-Phanephos nitrate (purified);

FIG. 12 shows the ³¹P {¹H}NMR spectrum of 1,2-Bis[(R,R)-2,5-diphenylphospholano]ethane;

FIG. 13 shows the ³¹P {¹H} NMR spectrum of 1,2-Bis[(R,R)-2,5-diphenylphospholano]ethane oxide;

FIG. 14 shows the ³¹P {¹H}NMR spectrum of the product of 1,2-Bis[(R,R)-2,5-diphenylphospholano]ethane oxide nitration (crude); and

FIG. 15 shows the ³¹P {¹H} NMR spectrum of 1,2-Bis[(R,R)-2,5-diphenylphospholano]ethane nitrate (partially purified).

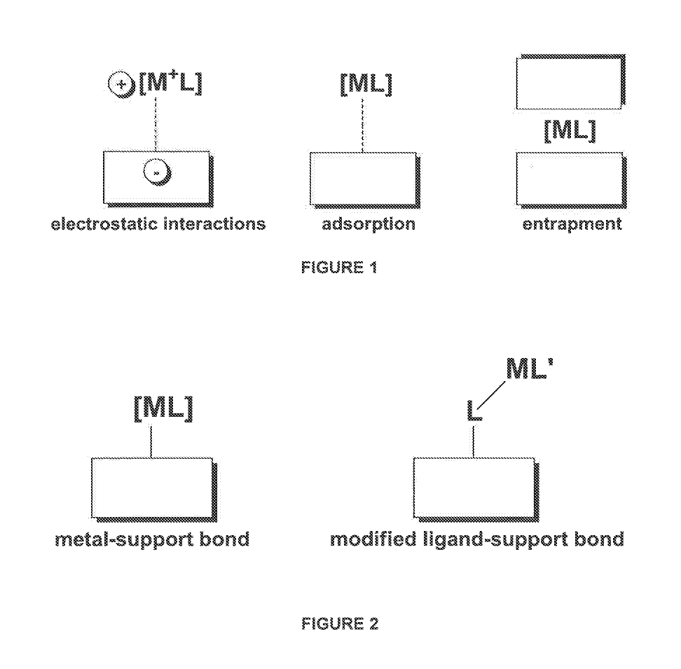

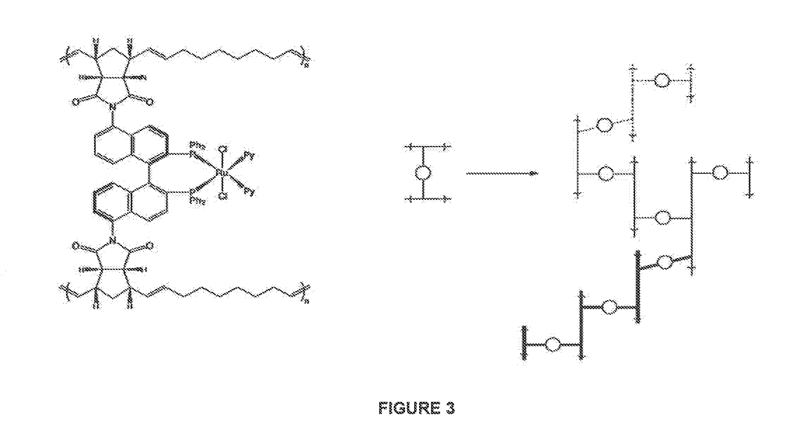

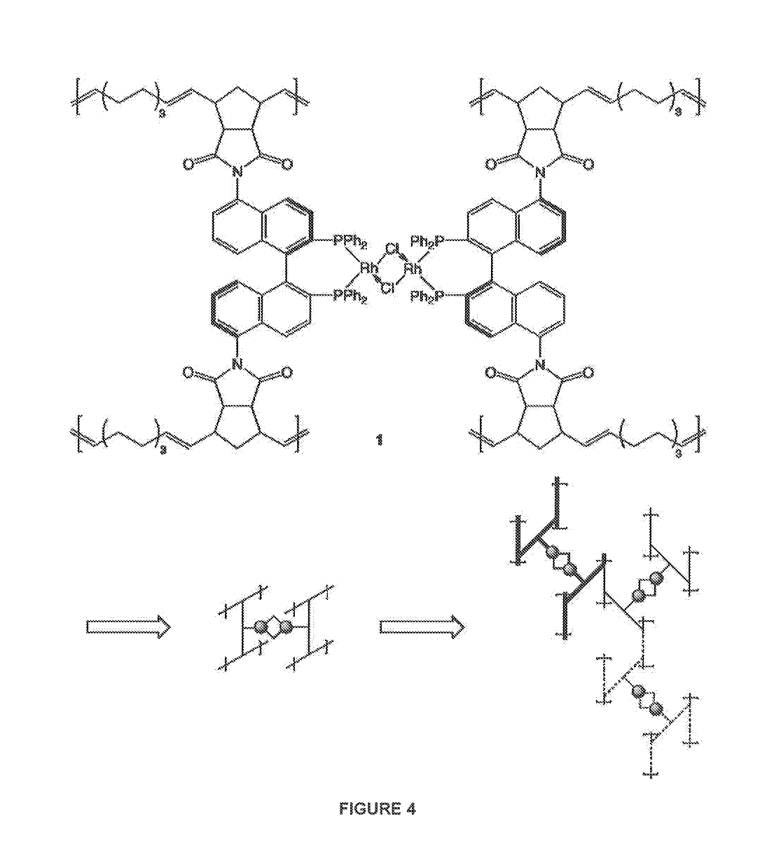

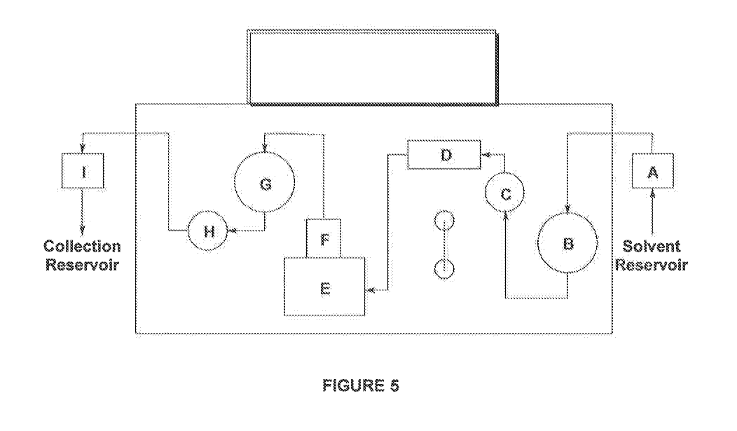

### DETAILED DESCRIPTION

Unless defined otherwise, all technical and scientific terms used herein have the same meaning as commonly understood by one of ordinary skill in the art to which this invention belongs.

As used in the specification and claims, the singular forms “a”, “an” and “the” include plural references unless the context clearly dictates otherwise.

As used herein, “aliphatic” refers to hydrocarbon moieties that are linear, branched or cyclic, may be alkyl, alkenyl, or alkynyl, and may be substituted or unsubstituted. “Alkyl” refers to a linear, branched or cyclic saturated hydrocarbon group. “Alkenyl” means a hydrocarbon moiety that is linear, branched or cyclic and contains at least one carbon to carbon double bond. “Alkynyl” means a hydrocarbon moiety that is linear, branched or cyclic and contains at least one carbon to carbon triple bond.

As used herein, “aryl” means a moiety including a substituted or unsubstituted aromatic ring, including heteroaryl moieties and moieties with more than one conjugated aromatic ring; optionally it may also include one or more non-aromatic ring. “C5 to C8 Aryl” means a moiety including a substituted or unsubstituted aromatic ring having from 5 to 8 carbon atoms in one or more conjugated aromatic rings. Examples of aryl moieties include phenyl.

“Alkylene” means a divalent alkyl radical, e.g., —C_{f}H_{2f}— wherein f is an integer. “Alkenylene” means a divalent alkenyl radical, e.g., —CHCH—. “Alkynylene” means a divalent alkynyl radical. “Arylene” means a divalent aryl radical, e.g., —C₆H₄—. “Heteroarylene” means a divalent heteroaryl radical, e.g., —O₅H₃N—. “Alkylene-aryl” means a divalent alkylene radical attached at one of its two free valencies to an aryl radical, e.g., —CH₂—C₆H₅. “Alkenylene-aryl” means a divalent alkenylene radical attached at one of its two free valencies to an aryl radical, e.g., —CHCH—C₆H₅. “Alkylene-heteroaryl” means a divalent alkylene radical attached at one of its two free valencies to a heteroaryl radical, e.g., —CH₂—C₅H₄N. “Alkenylene-heteroaryl” means a divalent alkenylene radical attached at one of its two free valencies to a heteroaryl radical, e.g., —CHCH—C₅H₄N—.

The term “comprising,” as used herein, will be understood to mean that the list following is non-exhaustive and may or may not include any other additional suitable items, for example one or more further feature(s), component(s) and/or ingredient(s) as appropriate.

The term “cycloalkyl,” as used herein, refers to a monocyclic, saturated carbocylic group, such as “C₄₋₈cycloalkyl” which, as used herein, means a monocyclic, saturated carbocylic group containing from four to eight carbon atoms and includes, but is not limited to, cyclobutyl, cyclopentyl, cyclohexyl, cyclopentyl and cyclooctyl.

“Heteroaryl” means a moiety including a substituted or unsubstituted aromatic ring having from 4 to 8 carbon atoms and at least one heteroatom in one or more conjugated aromatic rings. As used herein, “heteroatom” refers to non-carbon and non-hydrogen atoms, such as, for example, O, S, and N. Examples of heteroaryl moieties include pyridyl tetrahydrofuranyl and thienyl.

“Substituted” means having one or more substituent moieties whose presence does not interfere with the desired reaction. Examples of substituents include alkyl, alkenyl, alkynyl, aryl, heteroaryl, cycloalkyl (non-aromatic ring), alkoxyl, amino, alkylamino, alkenylamino, amide, amidine, hydroxyl, thioether, alkylcarbonyl, alkylcarbonyloxy, arylcarbonyloxy, alkoxycarbonyloxy, aryloxycarbonyloxy, alkoxycarbonyl, aminocarbonyl, alkylthiocarbonyl, imino, sulfhydryl, alkylthio, arylthio, thiocarboxylate, dithiocarboxylate, sulfate, sulfato, sulfonate, sulfamoyl, sulfonamide, nitro, nitrile, azido, heterocyclyl, ether, ester, ferrocenyl, silicon-containing moieties, thioester, or a combination thereof. The substituents may themselves be substituted.

As used herein, the term “unsubstituted” refers to any open valence of an atom being occupied by hydrogen. Also, if an occupant of an open valence position on an atom is not specified then it is hydrogen.

The term “halo,” as used herein means chloro, bromo, iodo or fluoro.

The term “monocyclic, bicyclic or tricylic ring system,” as used herein, refers to a carbon-containing ring system, that includes, but is not limited to, monocycles, fused and spirocyclic bicyclic and tricyclic rings, and bridged rings. Where specified, the carbons in the rings may be substituted or replaced with heteroatoms.

The term “linked to,” as used herein, means that referenced groups are joined via a linker group, which is a direct bond or an alkylene chain, in which the carbons in the chain are optionally substituted or replaced with heteroatoms.

The catalytic subunits as described herein optionally have at least one asymmetric centre. Where these compounds possess more than one asymmetric centre, they can exist as diastereomers. It is to be understood that all such isomers and mixtures thereof in any proportion are encompassed within the scope of the present application. It is to be understood that while stereochemistry of the compounds of the present application may be as shown for any given compound listed herein, such compounds may also contain certain amounts (for example less than 30%, less than 20%, less than 10%, or less than 5%) of corresponding compounds having alternate stereochemistry.

The term “suitable”, as in for example, “suitable anionic ligand” or “suitable reaction conditions” means that selection of a particular group or conditions would depend on specific synthetic manipulations to be performed, and the identity of the molecule, but said selection would be well within the skill of a person trained in the art. All process steps described herein are to be conducted under conditions suitable to provide a desired product(s). A person skilled in the art would understand that all reaction conditions, including, for example, reaction solvent, reaction time, reaction temperature, reaction pressure, reactant ratio and whether or not the reaction should be performed under an anhydrous or inert atmosphere, can be varied to optimize yield of a desired product(s), and it is within their skill to do so.

In some cases, the chemistries outlined herein may have to be modified, for instance by use of protecting groups, to prevent side reactions of reactive groups attached as substituents. This may be achieved by means of conventional protecting groups, for example as described in “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3.sup.rd Edition, 1999.

The terms “protective group” or “protecting group” or “PG” or the like as used herein refer to a chemical moiety which protects or masks a reactive portion of a molecule to prevent side reactions in those reactive portions of the molecule, while manipulating or reacting a different portion of the molecule. After manipulation or reaction is complete, the protecting group is removed under conditions that do not destroy or decompose the molecule. Many conventional protecting groups are known in the art, for example as described in “Protective Groups in Organic Chemistry” McOmie, J. F. W. Ed., Plenum Press, 1973 and in Greene, T. W. and Wuts, P. G. M., “Protective Groups in Organic Synthesis”, John Wiley & Sons, 3.sup.rd Edition, 1999. These may include but are not limited to Boc, Ts, Ms, TBDMS, TBDPS, Tf, Bn, allyl, Fmoc, C₁₋₁₆-acyl, silyl, and the like.

The term “intramolecular cycloisomerization” as used herein refers to a reaction wherein two or more functional groups in the same molecule react with each other to form a cyclic structure with the isomerization of one or more double or triple bonds.

The term “isomerization” as used herein refers to the process by which one molecule is transformed into another molecule that has exactly the same atoms, but the atoms are rearranged.

The term “flow reactor” as used herein refers to a dynamic reactor system in which reactants flow continuously into the vessel and products are continuously removed, in contrast to a batch reactor (as defined in McGraw-Hill Dictionary of Scientific & Technical Terms, 6E, Copyright© 2003 by The McGraw-Hill Companies, Inc.). Examples of flow reactors include, but are not limited to, continuous flow microreactors (e.g., the H-Cube® continuous flow hydrogenation reactor marketed by ThalesNano), fluidized bed reactors, membrane reactors laminar flow reactors, baffle flow reactors and the like.

The present application provides materials, systems and compositions for use in heterogeneous flow reactors. In particular, the present application provides a composite material containing a polymer-supported catalyst, or catalyst organic framework, immobilized on and/or in a solid support material. The polymer-supported catalyst comprises catalyst-containing monomer subunits incorporated in a polymer framework and each catalyst-containing monomer subunit comprises a transition metal covalently bound to a catalyst ligand.

Catalytic Polymeric Framework

It remains a challenge to reliably prepare polymeric chiral catalysts that are reliably reusable and that have activities comparable to the homogeneous systems from which they are derived. The composite material, system and method described herein incorporate a catalytic polymeric framework, where the framework comprises metal catalyst-containing monomeric units each separated by at least one non-catalyst containing monomeric unit. The framework can be formed by sequential polymerization of the constituent monomer subunits. Use of the covalently bonded polymeric framework has been shown to reduce the possibility of metal being leached from the integral catalytic monomeric unit during use, in comparison to other heterogeneous systems.

The catalytic polymeric framework can be prepared using various methods. For example, the catalyst monomer subunit can be modified to include polymerizable moieties so that the polymer framework can be prepared, and subsequently immobilized on a support material, via covalent or non-covalent interactions, to form a catalytic composite material (as described in more detail below). Alternatively, the support material itself can include polymerizable moieties so that it can participate in the formation of the framework as part of the composite material. This alternative results in covalent attachment of the catalytic polymeric framework to the support material.

In another embodiment, a polymeric framework can be prepared having groups suitable for grafting of catalyst subunits to produce the catalytic polymer framework.

In one example, preparation of the catalytic polymeric framework relies on a previously developed, versatile method to convert active and selective homogeneous catalysts into highly reusable, solid, catalyst-organic frameworks. For example, a Ru-BINAP framework was previously reported that, to the inventors' knowledge, provides the highest turnover number reuses of any chiral polymeric catalyst to date (Scheme 1). (Ralph, C. K., Bergens, S. H., Organometallics 2007, 26, 4)

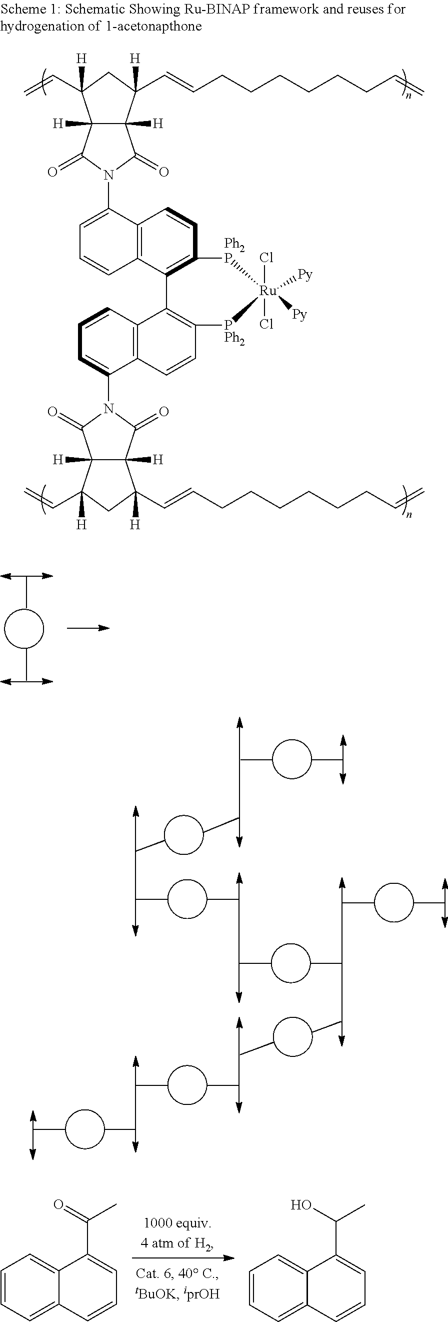

BINAP is a ubiquitous chiral ligand in asymmetric catalysis, and Ru is an active metal centre useful for hydrogenation of carbonyl compounds including ketones, esters, imines, imides, and recently, amides. For the production of catalytic polymeric frameworks, BINAP was modified with norimido groups at the 5,5′-positions (norimidobinap).

A process called alternating ROMP assembly (Scheme 2, ROMP is ring-opening olefin metathesis polymerization) has been used to prepare such catalytic polymeric frameworks. Briefly, norimido olefin groups attached to BINAP are strained, making them reactive towards ROMP. These norimido groups are also crowded, which prevents sequential, side-by-side polymerization. Consequently, during polymerization, a norimido group reacts with a metathesis catalyst (for example, a well-known first generation Grubbs Ru catalyst. Ru(Cl)₂(PCy₃)₂(═CHPh) has been successfully employed in this synthesis), to form an intermediate that is too crowded to react with another norimido group. Instead, it reacts with added cyclooctene (COE), which is less strained than the norimido group, but also less crowded. The result is insertion of a linear C₈-spacer to form an uncrowded intermediate that now reacts with another norimido group, and so on. The result is an alternating, three-dimensional catalytic polymeric framework with the catalyst acting as a cross linking agent. This synthesis has been proven to be versatile such that it has been applied to Ru, Rh, and Pd-BINAP systems; however, as would be well understood by a worker skilled in the art, these catalytic polymeric frameworks can incorporate any transition metal of interest.

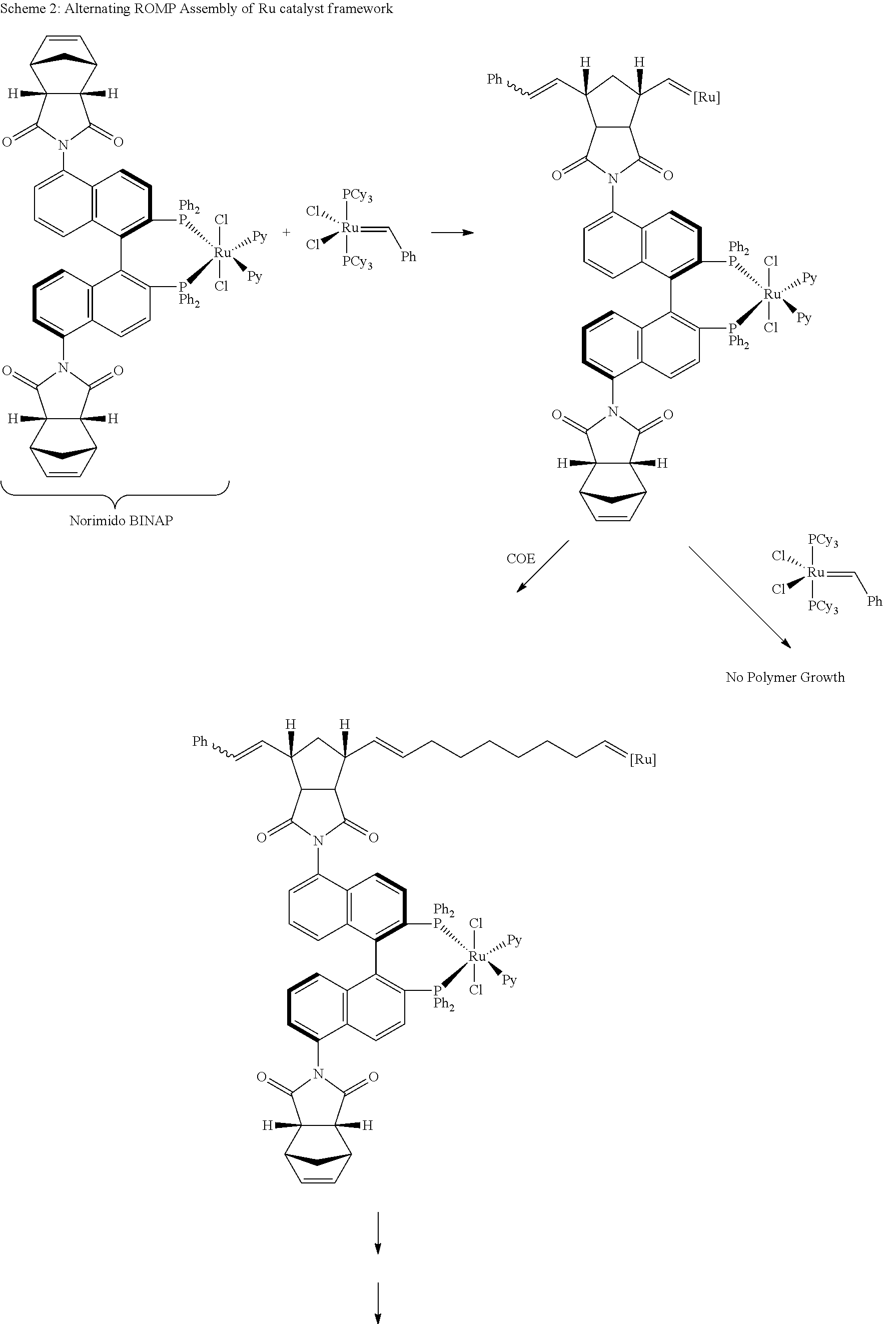

In recently published work (Bergens. S. H.; Sullivan, A. D.; Hass, M. Heterogeneous Rhodium Metal Catalysts. 2010), a Rh-norimidobinap framework was prepared using alternating ROMP assembly. This framework and its synthesis is also the subject of U.S. patent publication 2013/0053576, which is incorporated herein in its entirety.

Building from these previous systems, the present inventors have now found that similar methods can be employed to prepare catalytic polymeric frameworks incorporating various catalysts. In order for a catalyst to be incorporated into the polymeric framework, it must be included in a monomer that comprises the catalyst or catalyst ligand that has been modified to include polymerizable moieties. Preferably, the polymerizable moieties are strained and crowded, thereby making them suitable for alt-ROMP assembly with a linker monomer as described above, rather than side-by-side sequential polymerization.

In accordance with one embodiment, the catalyst-containing monomer has the structure:

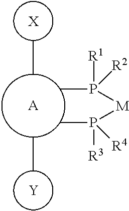

wherein

A is a substituted or unsubstituted aliphatic or aryl group;

X and Y are each independently a polymerizable moiety, wherein one of X or Y may be absent;

R¹, R², R³ and R⁴ are independently selected from aryl (e.g., phenyl), and C₄₋₈cycloalkyl, the latter two groups being unsubstituted or substituted, where possible, with 1, 2, 3, 4, or 5 groups independently selected from C₁₋₆alkyl, OC₁₋₆alkyl and halo, or R¹ and R² and/or R³ and R⁴ together with the atoms to which they are attached form a substituted or unsubstituted cycloalkyl; and

M is a transition metal (such as Ru, Rh, Pd, Pt, Ir, Fe, Ni or Co), optionally bound to another ligand or combination of ligands.

Examples of suitable polymerizable moeities include, but are not limited to:

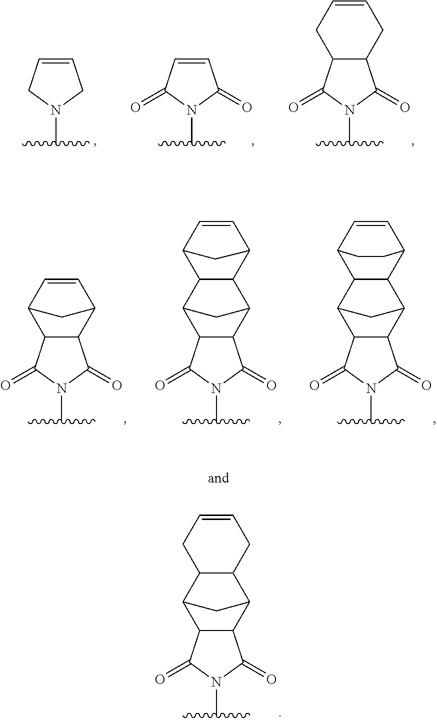

Selection of the specific catalyst to be used in the preparation of the catalyst-containing monomer is based on the reaction of interest to the user. In one example, the catalyst comprises a diphosphine ligand. In a specific embodiment, the catalyst-containing monomer is derived from a catalyst that comprises a ligand that is

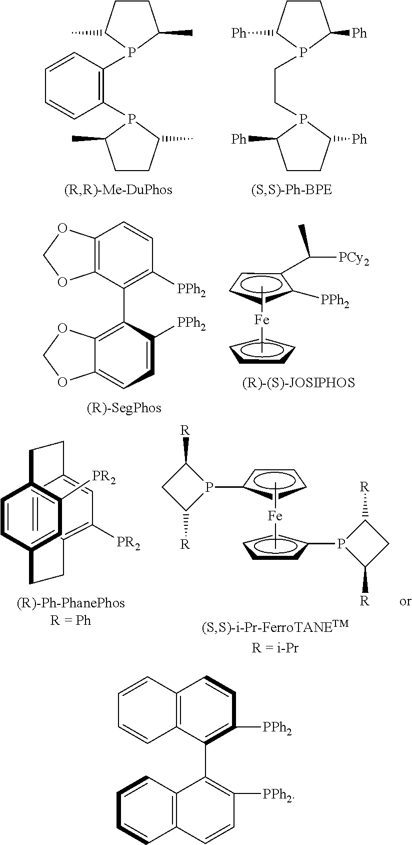

In certain embodiments, the catalyst-containing monomer does not comprise a BINAP ligand, or the polymerizable moiety does not comprise a norbornene.

In addition, to facilitate use of these catalytic polymeric frameworks in asymmetric catalysis, it is important that the catalyst monomer comprise at least one asymmetric centre.

In one embodiment, the catalytic polymeric framework comprises repeating catalyst-containing monomeric units of Formula I below:

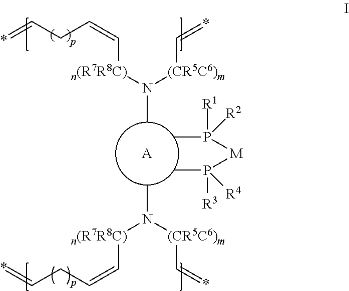

wherein

R¹, R², R³ and R⁴ are independently selected from aryl, such as phenyl, and C₄₋₈cycloalkyl, these groups being unsubstituted or substituted, where possible, with 1, 2, 3, 4, or 5 groups independently selected from C₁₋₆alkyl, OC₁₋₆alkyl and halo;

A is a binaphthyl group or a derivative of a binaphthyl group, each being unsubstituted or substituted with one or more groups independently selected from C₁₋₆alkyl, OC₁₋₆alkyl and halo;

R⁵, R⁶, R⁷ and R⁸ are independently selected from H, C₁₋₆alkyl, OC₁₋₆alkyl and halo; or R⁵ and R⁶ and/or R⁷ and R⁸ are ═O; or one of R⁵ and R⁶ is linked to one of R⁷ and R⁸ to form, together with the atoms to which they are attached and the atoms connecting them, a monocyclic, bicyclic or tricylic ring system; R⁵, R⁶, R⁷ and R⁸ in each methylene unit is the same or different, and means the double bond attached to this bond is in the cis or trans configuration, if applicable;

m and n are, independently, an integer between and including 0 and 10;

p is an integer between and including 1 and 14; and

M is a transition metal, optionally bound (e.g., coordinated) to a ligand.

In another embodiment, A is a binaphthyl group or a derivative of a binaphthyl group, each being unsubstituted or substituted with 1, 2, 3, 4, 5 or 6 groups independently selected from C₁₋₄alkyl, OC₁₋₄alkyl, chloro and fluoro. In another embodiment, is 1,1′-binaphthyl, 5,5′,6,6′,7,7′,8,8′-octahydro-1,1′-binaphthyl or 12,13,14,15,16,17,12′,13′,14′,15′,16′,17′-dodecahydro-11H,11′H-[4,4′]bi[cyclopenta[a]phenanthrenyl], each being unsubstituted or substituted with 1, 2, 3, 4, 5 or 6 groups independently selected from C₁₋₄alkyl, OC₁₋₄alkyl, chloro and fluoro. In another embodiment, A is optically active.

In accordance with certain embodiments, transition metal M is Ru, Rh, Pd, Pt, Ir, Fe, Ni or Co.

The system and composite material described herein can be readily modified to incorporate catalytic monomers that are based on a variety of homogeneous catalysts. Such catalysts can need to be modified by incorporation of polymerizable moeities so that they can be polymerized, for example, via altROMP. For example, additional rhodium based catalyst monomers can be prepared based on a versatile homogeneous hydrogenation catalyst, [Rh(COD)₂]BF₄+2 L system, where L is a monodentate phosphoramidite ((BINOL)P(NR2)) or phosphite (BINOL)P(OR) developed by DeVris et al. (de Vries, A. H. M.; Meetsma, A.; Feringa, B. L. Angew. Chem. Int. Ed. 1996, 35, 2374; and Hulst, R.; de Vries, K.; Feringa, B. L. Tetrahedron: Asymmetry 1994, 5, 699). This system has produced homogeneous Rh catalysts that hydrogenate a wide number of imines, enol acetates, itaconic acids, α- and β-dehydroamino acids and esters, and other prochiral olefins in high ee. Further, these ligands provide high ee for a large number of catalytic reactions besides hydrogenation (Minnaard, A. J.; Feringa, B. L.; Lefort, L.; de Vries, J. G. Acc. Chem. Res. 2007, 40, 1267). The altROMP methodology can be used to prepare heterogeneous analogs of selective and versatile homogeneous [Rh((3,3′-R′-BINOL)P(X))₂(COD)](BF₄) (R′=H, Me, X=secondary amine or alkoxide) catalysts reported in literature for hydrogenations.

In another embodiment, the system and methods described herein can be used to prepare additional ruthenium based catalyst systems. Ru-BINAP-based catalysts are active and are highly enantioselective for olefin, keto-ester, ketone, and imine hydrogenations. In published work, (Wiles, J. A.; Daley, C. J. A.; Hamilton, R. J.; Leong, C. J.; Bergens, S. H. Organometallics 2004, 23, 4564) it has been shown that [Ru(BINAP)(η₅-C₈H₁₁)]+(BF₄—) is an active and selective olefin hydrogenation catalyst. In another publication, (Akotsi, O. M., Metera, K., Reid, R. D. McDonald, R., Bergens, S. H. Chirality 2000, 12, 514-522), it has been shown that Ru(5,5′-BINAP)(py)₂(Cl)₂ is active and selective for hydrogenation of ketoesters. 5,5′-dinoramido-BINAP versions of the catalytic polymeric framework can be prepared and incorporated into flow reactor cartridges for hydrogenations of prochiral olefins, ketoesters, and related substrates.

In another embodiment, the system and methods described herein can be used to prepare iron based catalyst systems. It has been reported that Fe(P—N—N—P) complexes are active for selective ketone hydrogenations (Prokopchuk, D. E.; Morris, R. H. Organometallics 2012, 31, 7375). Being based on iron, these catalysts are generally considered “greener” than competitive catalysts comprising heavy metals. Analogous versions of these catalysts that are active toward altROMP can be prepared for use in manufacture of a heterogeneous flow system, as described herein, through the incorporation of polymerizable moieties into the catalyst ligand.

In one aspect, there is provided a method of preparing a catalyst-containing monomer for incorporation into a catalytic polymeric framework as described herein. The method comprises the step of adding one or more polymerizable moieties to the ligand of the catalyst to be incorporated into the polymeric framework. In one example, this step comprises nitrating the ligand at one or more positions, reducing the resulting nitrated ligand to generate one or more amines, which are amenable to derivatization for attachment of the polymerizable moiety to the catalyst ligand. In the case where only one polymerizable moiety is incorporated into the catalyst-containing monomer, the resulting polymeric framework comprises a linear framework. In the cases in which more than one polymerizable moiety is incorporated into the catalyst-containing monomer, the resulting polymeric framework comprises a crosslinked framework.

In a related aspect, there is provided a method of preparing a catalytic polymeric framework comprising the steps of: (i) adding one or more polymerizable moieties to the ligand of the catalyst to be incorporated into the polymeric framework to form a catalyst-containing monomer; and (ii) polymerizing the catalyst-containing monomer with a non-catalyst-containing monomer. As described above, the polymerizing step can be an alternating ring-opening polymerization, in which case both the polymerizable moiety and the polymerizable moiety of the non-catalyst-containing monomer comprise a ring (or cycle). Non-limiting examples of suitable polymerizable moieties are provided above. Furthermore, selection of a suitable non-catalyst-containing monomer would be a matter of routine to a worker skilled in the art.

Catalytic Composite Materials

The catalytic polymeric frameworks described above have now been found to be particularly useful in the manufacture of composite materials suitable for use in catalytic flow reactors.

As described above, the catalytic polymeric framework can be prepared using various methods. In the example in which the catalyst monomer subunit is modified to include polymerizable moieties in order to facilitate the manufacture of the polymer framework, the resulting polymer framework can be subsequently immobilized on a suitable support material, via covalent or non-covalent interactions, to form the catalytic composite material. Similarly, in the example in which the catalytic polymer framework is prepared by grafting of the catalytic subunits into the framework, the resulting polymer framework can be subsequently immobilized on a suitable support material, via covalent or non-covalent interactions, to form the catalytic composite material.

Alternatively, the support material itself can include polymerizable moieties so that it can participate in the formation of the framework as part of single pot manufacture of the composite material. This alternative results in covalent attachment of the catalytic polymeric framework to the support material.

In one example, the catalytic composite material is generally prepared by combining a catalytic polymeric framework with an appropriate solid material under conditions suitable for adherence or attachment of the polymeric framework to the solid material. Selection of the appropriate solid material is dependent, at least in part, on the type of flow reactor system intended for use.

As described above, and as is well known to those of skill in the art, flow reactors facilitate chemical reactions in such a manner that reactants can be continuously added to the reactor as products are removed. The use of a catalytic solid support material in such reactor systems means that the catalyst does not need to be continually added to and retrieved from the reactor flow. Flow reactors can employ various forms of catalytic solid support materials, such as, for example, beads, powders, membranes and the like. The materials used in these materials can vary depending on the type of reactor and the form of support material. Non-limiting examples of suitable support materials include BaSO₄, barium (L)- and (D)-tartrates, aluminum oxide (Al₂O₃), silica (SiO₂), Fe₃O₄, Teflon™, Celite™, AgCl and sand.

Although the present application focuses on the manufacture and use of the catalytic composite materials in flow reactor systems, such composite materials can also be used in batch reactor systems.

Continuous Flow Systems

Within the last 20 years, requirements for environmentally friendly and sustainable chemical processes has increased, due, in part, to concerns regarding negative impacts of industry on the environment. Specifically, environmentalists have been focused on minimizing industrial pollution and waste. As a result of these concerns, industry has been attempting to reduce chemical waste, maximize atom economy and increase production, all while minimizing total energy input, utilizing safe chemical processes and maximizing catalytic efficiency. As a result of this initiative, a significant amount of research has been focused on developing continuous-flow catalytic reactors and processes that can be applied to industrial-scale preparations.

Although often requiring time intensive initial equipment set-up and optimization of concentrations, temperatures, pressures and flow rates, continuous-flow catalytic processes have potential to address many environmental and industrial demands, as mentioned above.

In addition to designing and adapting catalysts for continuous-flow processes, there has been a significant amount of research focused on development of flow reactors themselves. Common lab scale flow reactors include, but are not limited to, (a) fixed-bed reactors, where immobilized catalysts are fixed in, and a flowing substrate occupies vacancies between catalyst particles; (b) trickle-bed reactors, where, in a downward movement, a particular substrate is allowed to move over a packed bed of immobilized catalyst particles; and (c) tube reactors, where a homogeneous catalyst, combined with a substrate, is pumped through a tubular column of varying length to an outlet valve.

Recently, Thales Nanotechnology® reported development of a commercially available continuous-flow reactor. The reactor, named H-Cube®, combines hydrogen, generated from electrolysis of water, with a continuous-flow system, resulting in efficient hydrogenations of numerous substrates catalyzed by a variety of commercially available, immobilized catalysts. A schematic of the H-Cube® Is shown in FIG. 5.

As shown in FIG. 5, solvent, or a substrate solution, is delivered to the H-Cube® through an HPLC pump A. Once the solution enters the reaction line, it is passed through an inlet pressure sensor B, and is combined with generated hydrogen in a substrate/hydrogen mixer, C. Next, the gas/solution mixture is passed through a bubble detector D, which determines if there is hydrogen in the reaction line, and then into a catalyst cartridge (CatCart®) heating unit E. The CatCart® itself (F) contains an immobilized catalyst and is situated within the CatCart® heating unit E. It should be noted that in addition to providing a variety of pre-packed CatCarts®, Thales Nanotechnology® also supplies empty CatCarts® allowing users to test their own immobilized catalysts in the H-Cube®. After the gas/solution mixture is exposed to the immobilized catalyst, it flows out of the CatCart® F and through an outlet pressure sensor G, and a back-pressure regulator H. The back-pressure regulator H can restrict flow of solvent/substrate through the system to maintain a desired hydrogen pressure throughout. Finally, the solution exits the H-Cube® through a hydrogenated product collector I, and enters a collection reservoir.

The H-Cube®, like any other continuous-flow reactor, provides benefits over traditional batch reactors found in industry. In addition, the H-Cube® generates hydrogen through electrolysis of water, thus removing any need for a hydrogen cylinder. As well, all of the generated hydrogen is used in situ, preventing any unsafe build-up of hydrogen pressure within the instrument.

In one embodiment, the above-described catalytic polymeric framework is introduced into a continuous-flow reactor column (or cartridge). In another embodiment, the cartridge is suitable for use in the H-Cube®.

Studies performed using a poly-[Rh(NBD)((R)-5,5′-dinorimido-BINAP)](SbF₆)/BaSO₄ catalytic polymeric framework packed in an H-Cube® cartridge are described in detail in the following Examples (note, NBD is norbornadiene). These studies have demonstrated the use of such catalytic polymeric framework sin various hydrogenation reactions. Overall results of these studies are listed in Table 1 below:

TABLE 1
Summary of the longevity and total TONs obtained
from CatCarts ® loaded with the rhodium catalytic
polymeric framework_42

| | Longevity^{a} | | # of Different |
|---|---|---|---|
| Entry | (days) | Total TONs | Substrates Tested |
| 1 | 25 | 36,500 | 7 (71, 73, 74, 75, MAA, 100, 102) |
| 2 | 30^{b} | 55,700 | 3 (71, MAA, 88) |
| 3 | 27^{b} | 17,600 | 2 (71, 77) |

^{a}The longevity refers to the number of consecutive days that the catalyst was present in the H-Cube ® and remained active. After the indicated period of time, the catalyst was removed from the H-Cube ® and was not used in any further catalytic experiments.
^{b}The catalyst was still active upon removal from the H-Cube ®.

Additional studies performed using Poly-[RhCl((R)-5,5′-dinorimido-BINAP)]₂/Ba-L-Tartrate catalytic polymeric framework 41 in the H-Cube® again demonstrated a successful use of this catalyst system. In this case, although overall yields were lower than observed using the poly-[Rh(NBD)((R)-5,5′-dinorimido-BINAP)](SbF₆)/BaSO₄ catalytic polymeric framework, the ee obtained in each case was >99.9%. Thus, this catalyst was more selective, but a little less active than the BaSO₄-supported catalysts.

To gain a better understanding of the invention described herein, the following examples are set forth. It should be understood that these examples are for illustrative purposes only. Therefore, they should not limit the scope of this invention in any way.

### EXAMPLES

### General Procedures and Methods

Gas chromatography analyses were carried out using a Hewlett-Packard 5890 chromatograph equipped with a flame ionization detector, a 3392A integrator, and a Supelco Beta Dex™ 120 fused silica capillary column (30 m×0.25 mm×0.25 μm). HPLC analyses were performed using a Waters 600E multisolvent delivery system equipped with Waters 715 Ultra WISP sample processor, Waters temperature control system, Waters 990 photodiode array detector, Waters 410 differential refractometer, Waters 5200 printer plotter, and Daicel CHIRALPAK IB (4.6 mm i.d.×250 mm) chiral column. HPLC grade hexanes (Min. 99.5%) and 2-propanol (Min. 99.5%) were obtained from Caledon Laboratories Ltd. Continuous-flow reactions were performed using an H-Cube® SS continuous-flow hydrogenation reactor equipped with a K-120 HPLC pump. CatCarts® and related packing products were obtained from ThalesNano Nanotechnology Inc.

Unless otherwise stated, all experiments were performed under an inert atmosphere using standard Schlenk and glove-box techniques. Argon and nitrogen gas (Praxair, 99.998%) were passed through a drying train containing 3 Å molecular sieves and indicating Drierite™ before use. All solvents were dried and distilled under a nitrogen atmosphere using standard drying agents, unless otherwise noted. All allylic alcohol reagents and dimethyl itaconate were obtained from Sigma-Aldrich Co. and were distilled under a nitrogen atmosphere prior to use. Methyl α-acetamido acrylate and itaconic acid were obtained from Sigma-Aldrich Co. and used without further purification. α-Acetamidocinnamic acid was synthesized according to literature procedures. (Shinkai, H.; Toi, K.; Kumashiro, I.; Seto, Y.; Fukuma, M.; Dan, K.; Toyoshima, S. J. Med. Chem. 1988, 31, 2092).

### Synthesis of (R)-5,5′-dinorimido-BINAP (N-BINAP)

(Ralph, C. K.; Bergens, S. H. Organometallics, 2007, 26, 1571-1574, (b) Corkum, E. G.; Hass, M. J.; Sullivan, A. D.; Bergens, S. H. Org. Lett. 2011, 13, 3522-3525)

(R)-5,5′-diamino-BINAP (Okano, T. K. H.; Akutagawa, S.; Kiji, J.; Konishi, H.; Fukuyama, K.; Shimano, Y. U.S. Pat. No. 4,705,895, 1987.) (0.77 g, 1.179 mmol), a known precursor, was added to a thick walled Schlenk flask. The flask was evacuated and back-filled three times with nitrogen gas and then sealed with a rubber septum. A large excess (12 eq.) of cis-5-norbornene-endo-2,3-dicarboxylic anhydride (2.32 g, 14.156 mmol) was weighed into a 100 mL round-bottom flask equipped with a side-arm and evacuated and back-filled three times with nitrogen gas. The anhydride was dissolved in 25 mL of distilled, deoxygenated toluene and then transferred via cannula to the Schlenk flask containing the (R)-5,5′-diamino-BINAP to give a dark brownish-red colored solution. A large excess (12 eq.) of tripropylamine (2.02 g, 14.156 mmol) was then added to the Schlenk flask and the flask was sealed with a Teflon® valve. The solution was then stirred at 90° C. for 72 hours, during which a brown solid was observed in the flask. Next, the reactor was cooled to room temperature and the mixture was transferred via cannula to a purged 500 mL round-bottom flask equipped with a side-arm followed by 3×15 mL rinses with distilled, deoxygenated toluene. The solution was then treated with 100 mL of deoxygenated 1M aqueous NaOH. The yellowish-brown organic layer was then extracted with 3×15 mLs of toluene and transferred via cannula into a purged 500 mL round-bottom flask equipped with a side-arm that contained anhydrous NaSO₄. The solution was left to dry for approximately 1 hour. The toluene solution was then cannula filtered into a new purged 500 mL round-bottom flask equipped with a side-arm and the volatiles were removed via a secondary cold trap under high-vacuum to yield a brown solid of (R)-5,5′-dinorimido-BINAP (60% yield, 0.66 g, 0.698 mmol). Spectroscopic data was in accordance with literature. (Ralph, C. K.; Bergens, S. H. Organometallics, 2007, 26, 1571-1574, (b) Corkum, E. G.; Hass, M. J.; Sullivan, A. D.; Bergens, S. H. Org. Lett. 2011, 13, 3522-3525)

### Synthesis of BaSO₄ supported poly-[Rh(NBD)(N-BINAP)](SbF₆)

### Synthesis of [Rh(NBD)(N-BINAP)](SbF₆)

Under a nitrogen gas atmosphere, a solution of 79.0 mg (8.36×10⁻² mmol) of rotamerically pure N-BINAP in 0.7 mL of CD₂Cl₂ was transferred via cannula to a Schlenk flask containing 43.9 mg (8.36×10⁻² mmol) of [Rh(NBD)₂](SbF₆), giving a brown colored solution. The N-BINAP was rinsed into the Schlenk flask with an additional 0.3 mL of CD₂CL after which the flask was sealed and stirred at room temperature for 24 hours. ¹H and ³¹P-NMR were in accordance with the literature. (LaRocque, L. P.-A. (2008). Polymerization and Use of Rhodium and Ruthenium Catalysts for the Cycloisomerization Alder-Ene Reaction. M. Sc. Thesis. University of Alberta: Canada.)

### Synthesis of poly-[Rh(NBD)(N-BINAP)](SbF₆)

A cationic, NBD-containing precursor was prepared and subsequently polymerized into a framework as outlined in the following scheme. Preparations and polymerizations of these compounds all went in high yields and with good product purity,

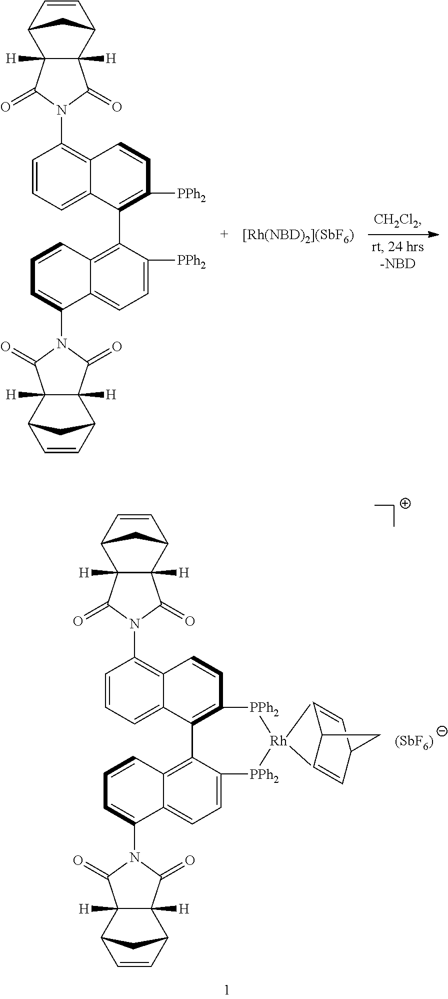

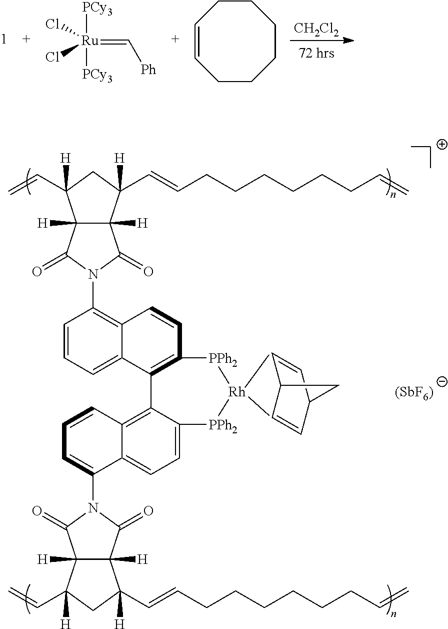

In a typical experiment, 24.6 mg (1.79×10⁻² mmol) of [Rh(NBD)(N-BINAP)](SbF₆) was dissolved in 0.5 mL of CH₂Cl₂ and transferred via cannula to a purged Schlenk flask. Under a nitrogen gas atmosphere, 14 μL (1.074×10⁻¹ mmol) of cis-cycloocetene was added to the Schlenk flask and rinsed in with 1.25 mL of CH₂Cl₂. Next, 0.7 mg (8.95×10⁻⁴ mmol) of trans-RuCl₂(PCy₃)₂(═CHPh) (Grubbs' 1^{st} Generation catalyst) was dissolved in 0.5 mL of CH₂Cl₂, yielding a purple solution. This solution was then transferred via cannula, under a nitrogen gas atmosphere, into the Schlenk flask. The vessel was then sealed and placed, with moderate stirring, into an oil bath at 45° C. for 72 hours. After 72 hours, an aliquot of the mixture was taken and the recorded NMR spectra confirmed that polymerization was complete. The spectroscopic data was in accordance with the literature.⁶ This mixture was then diluted with 10 mL of CH₂Cl₂.

### Deposition of poly-[Rh(NBD)(N-BINAP)](SbF₆) on BaSO₄

10 g of BaSO₄ was washed consecutively with 4×50 mL of CH₂Cl₂ followed by 3×50 mL of MeOH and then dried under vacuum at room temperature overnight.

2.592 g of the washed and dried BaSO₄ was weighed into a 250 mL round-bottom flask, equipped with a side-arm and a stir bar, and was evacuated and back-filled with nitrogen gas three times. 15 mL of CH₂Cl₂ was added to the flask and was stirred slowly to create a BaSO₄ slurry. The reaction mixture that contained the poly-[Rh(NBD)(N-BINAP)](SbF₆) prepared above was transferred via cannula, under a nitrogen gas atmosphere, into the flask containing the BaSO₄/CH₂Cl₂ slurry, creating a light brown colored mixture. The poly-[Rh(NBD)(N-BINAP)](SbF₆) was followed by 3×5 mL rinses of CH₂Cl₂ and the final slurry was stirred for 1 hour at room temperature to ensure an even distribution of poly-[Rh(NBD)(N-BINAP)](SbF₆) on the BaSO₄. The solvent was then slowly removed via a secondary cold trap under high-vacuum. After removal of the solvent to dryness, the solid product was dried further under high-vacuum for 1 hour. After the initial drying, the BaSO₄ supported poly-[Rh(NBD)(N-BINAP)](SbF₆) was rinsed with 3×20 mL of distilled, deoxygenated MeOH to remove any polymerized cis-cyclooctene and low molecular weight polymer. The pale yellow MeOH portions were cannula filtered under a nitrogen gas atmosphere into a round-bottom flask. After the final MeOH rinse, the catalyst was dried under high-vacuum for ˜2 hours then immediately transferred to the glove-box where it was stored until needed. NMR spectra recorded in CD₂Cl₂ of the MeOH residue showed only polymerized cis-cyclooctene present. There was also no observable signal in the ³¹P-NMR spectrum. Final loading of rhodium was 9.49 mg per gram of BaSO₄ support.

### Representative Procedure for Packing a CatCart® with the poly-[Rh(NBD)((R)-5,5′-dinorimido-BINAP)](SbF₆)BaSO₄ Catalytic Polymeric Framework (42)

An empty CatCart® (30×4 mm) was brought into the glove box and weighed (8.5267 g). In ˜50 mg increments, the BaSO₄ supported poly-[Rh(NBD)(N-BINAP)](SbF₆) was added to the empty CatCart®) via scoopula. After each addition of catalyst, the CatCart® was tapped for ˜3 minutes to ensure that all of the catalyst added was tightly and evenly packed in the CatCart®. Once the level of the catalyst reached the lip of the CatCart® (slightly below where the CatCart® “top” would be placed) no more catalyst was added and the full CatCart® was then weighed (8.9491 g, 0.4215 g of BaSO₄ supported catalyst in the CatCart®). The final loading of rhodium in the CatCart® was 4.16 mg (9.88 mg of rhodium per gram of BaSO₄ support). The packed CatCart® was stored in the glove box until required.

### Synthesis of Ba-L-Tartrate supported poly-[Rh(N-BINAP)Cl]₂

### Synthesis of [Rh(N-BINAP)Cl]₂

Under a nitrogen gas atmosphere, a solution of 11.4 mg (1.21×10⁻² mmol) of rotamerically pure N-BINAP in 0.5 mL of CD₂Cl₂ was added to a slurry of 2.3 mg (6.03×10⁻³ mmol) [Rh(C₂H₄)₂Cl]₂ in 0.1 mL of CD₂Cl₂ in an NMR tube. The NMR tube was shaken, and occasionally purged with nitrogen gas, for 30 minutes before ¹H-NMR and ³¹P-NMR spectra were obtained. Upon addition of the N-BINAP solution to the [Rh(C₂H₄)₂Cl]₂ slurry, there was a rapid color change from yellow-orange to brick red, with accompanying evolution of ethylene gas. After identification by NMR, the compound was used immediately and without isolation as attempts at isolation resulted in decomposition of the product. Spectroscopic data was in accordance with literature. (Corkum, E. G.; Hass, M. J.; Sullivan. A. D.; Bergens, S. H. Org. Lett. 2011, 13, 3522-3525; and Corkum, E. G.; Kalapugama, S.; Hass, M. J.; Bergens, S. H. RSC Advances 2012, 2, 3473-3476.)

### Synthesis of poly-[Rh(N-BINAP)Cl]₂

In a typical experiment, 13.1 mg (6.05×10⁻³ mmol) of [Rh(N-BINAP)Cl]₂ was prepared in 0.6 mL of CD₂Cl₂ in an NMR tube as described above. Under a nitrogen gas atmosphere, 9.5 μL of cis-cyclooctene (7.25×10² mmol) was added to the solution and the tube was shaken. The color of the solution remained brick red. This solution was then transferred via cannula, under a nitrogen gas atmosphere, into a purged Schlenk flask equipped with a stir bar, and rinsed in with 0.5 mL of CD₂Cl₂. Next, 0.5 mg (6.05×10⁻⁴ mmol) of trans-RuCl₂(PCy₃)₂(═CHPh) (Grubbs' 1^{st} Generation catalyst) was dissolved in 0.5 mL of CD₂Cl₂, yielding a purple solution. This solution was then transferred via cannula, under a nitrogen gas atmosphere, into the Schlenk flask. The vessel was then sealed and placed, with moderate stirring, into an oil bath at 40° C. for 24 hours. After 24 hours, an aliquot of the mixture was taken and the recorded NMR spectra confirmed that polymerization was complete. Spectroscopic data was in accordance with literature. (Corkum, E. G.; Hass, M. J.; Sullivan, A. D.; Bergens, S. H. Org. Lett. 2011, 13, 3522-3525; and LaRocque, L. P.-A. (2008). Polymerization and Use of Rhodium and Ruthenium Catalysts for the Cycloisomerization Alder-Ene Reaction. M. Sc. Thesis. University of Alberta: Canada) This mixture was then diluted with 10 mL of CH₂Cl₂.

The following scheme depicts the formation of the poly-[Rh(N-BINAP)Cl]2.

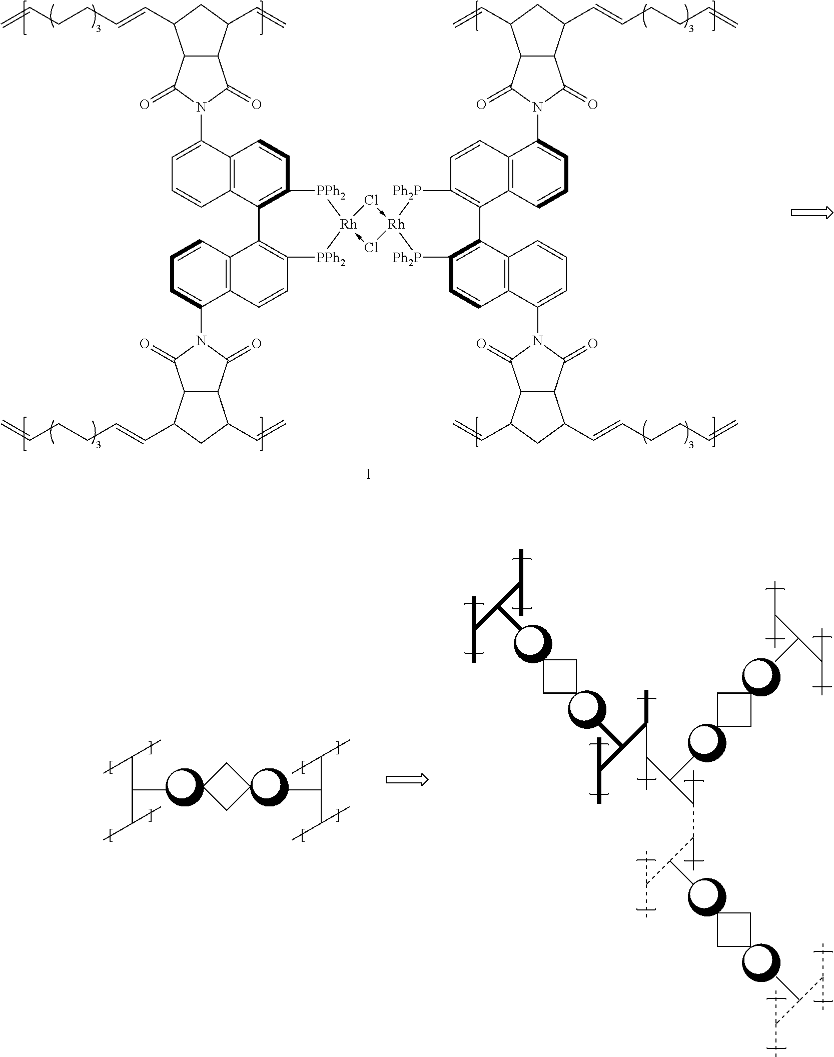

### Deposition of poly-[Rh(N-BINAP)Cl]₂ on Ba-L-Tartrate

10 g of Ba-L-Tartrate was washed consecutively with 4×50 mL of CH₂Cl₂ followed by 3×50 mL of MeOH and then dried under vacuum at room temperature overnight.

1.106 g of the washed and dried Ba-L-Tartrate was weighed into a 250 mL round-bottom flask, equipped with a side-arm and a stir bar, and was evacuated and back-filled with nitrogen gas three times. 15 mL of CH₂Cl₂ was added to the flask and was stirred slowly to create a Ba-L-Tartrate slurry. The reaction mixture that contained the poly-[Rh(N-BINAP)Cl]₂ prepared above was transferred via cannula, under a nitrogen gas atmosphere, into the flask containing the Ba-L-Tartrate/CH₂Cl₂ slurry, creating a tan-colored mixture. The poly-[Rh(N-BINAP)Cl]₂ was followed by 3×5 mL rinses of CH₂Cl₂ and the final slurry was stirred for 1 hour at room temperature to ensure an even distribution of poly-[Rh(N-BINAP)Cl]2 on the Ba-L-Tartrate. The solvent was then slowly removed via a secondary cold trap under high-vacuum. After removal of the solvent to dryness, the solid product was dried further under high-vacuum for 1 hour. After the initial drying, the Ba-L-Tartrate supported poly-[Rh(N-BINAP)Cl]₂ was rinsed with 3×20 mL of distilled, deoxygenated MeOH to remove any polymerized cis-cyclooctene and low molecular weight polymer. The MeOH portions were cannula filtered under a nitrogen gas atmosphere into a round-bottom flask. After the final MeOH rinse, the catalyst was dried under high-vacuum for ˜2 hours then immediately transferred to the glove-box where it was stored until needed. NMR spectra recorded in CD₂Cl₂ of the MeOH residue showed only polymerized cis-cyclooctene present. There was also no observable signal in the ³¹P-NMR spectrum. Final loading of rhodium was 11.74 mg per gram of Ba-L-Tartrate support.

### Representative procedure for packing a CatCart® with the poly-[RhCl((R)-5,5′-dinorimido-BINAP)]₂/Ba-L-Tartrate catalytic polymeric framework (41)

An empty CatCart® (30×4 mm) was brought into a glove box and weighed (8.4475 g). AgSbF₆ (0.0169 g, 4.92×10-2 mmol) was added initially to the CatCart® and the CatCart® was tapped for ˜3 minutes to ensure even packing. Next, AgSbF₆ (0.0109 g, 3.17×10-2 mmol) was mixed evenly with the Ba-L-Tartrate supported poly-[Rh(N-BINAP)Cl]₂. The catalyst/AgSbF₆ mixture was then added to the CatCart® via scoopula in ˜50 mg increments. After each addition of catalyst, the CatCart® was tapped for ˜3 minutes to ensure that all of the catalyst added was tightly and evenly packed in the CatCart®. Once the level of the catalyst reached the lip of the CatCart® (slightly below where the CatCart® “top” would be placed) no more catalyst was added and the full CatCart® was then weighed (8.7362 g, 0.2609 g of Ba-L-Tartrate supported catalyst in the CatCart®). Final loading of rhodium in the CatCart® was 3.09 mg (11.84 mg of rhodium per gram of Ba-L-Tartrate support). Final number of equivalents of AgSbF₆ per rhodium center was 25.5 equivalents. The packed CatCart® was stored in a glove box until required.

In the following studies four catalyst cartridges were used. Three (cartridges #1-3) had [Rh(BINAP)(NBD)]⁺ (SbF₆⁻) as the cross-linking polymer unit and with BaSO₄ as support. These columns were activated by H₂ in solution, which hydrogenates the NBD groups to expose an active catalyst, [Rh(BINAP)(sol)₂], where sol=solvent, reactant, or support. The fourth column (cartridge #4) had the neutral chloro-bridged dimer (Rh(BINAP)Cl)/₂ as an active site and was supported on barium (L)-tartrate. Structure of this framework is different from the other three frameworks because the active site has two Rh centres that are bridging two strands of the framework. The fourth catalyst was prepared in order to investigate whether having another Rh(BINAP) unit improves ee of hydrogenations, and whether pore size within this framework is larger. Also, this catalyst is supported on a chiral support (Ba (L)-tartrate), and it is anticipated that this added source of chirality improves the ee of these hydrogenations. This cartridge was activated by AgSbF₆ as described below. Results from use of each of these cartridges are summarized in the next sections.

### Representative Procedure for Pressing a Packed CatCart®) Loaded with a Particular Rhodium Catalytic Polymeric Framework

Packed CatCarts® were removed from a glove box for pressing. The packed CatCart® opening was covered first with a piece of pre-cut filter paper, followed by a pre-cut metal screen. Next, a rubber o-ring followed by a thick rubber o-ring were placed on top of the metal screen. The thick rubber o-ring was pressed down slightly with tweezers to keep all the components in place for pressing. Using an arbor press, the components were pressed into the CatCart® thus sealing the contents. The CatCart® was then immediately transferred to the H-Cube® CatCart® holder for use.

### Representative Procedure for Operating the H-Cube

A packed and pressed CatCart® was inserted into the H-Cube® CatCart® holder and the H-Cube® water reservoir was filled with triply distilled water. The solvent and substrate were freshly distilled and bubbled with nitrogen gas for 30 minutes prior to use in the H-Cube®. A substrate solution of desired concentration was prepared in a purged round-bottom flask equipped with a side-arm.

In a typical experiment, the H-Cube® and connected HPLC pump were switched on. The H-Cube® water line was then purged for ˜1 minute, followed by a purging of the HPLC pump inlet with a desired solvent to remove and prevent any air bubbles from entering the pump itself. Next, desired parameters (i.e. temperature, H₂ pressure and flow rate) were programmed into the H-Cube® using the H-Cube® interface. The HPLC pump was then initiated and pure solvent was flushed through the H-Cube for ˜10 minutes. The H-Cube® was then started and internal pressures were allowed to build-up and stabilize over the course of ˜10 minutes. Once the system was stable, pure H₂ and solvent were flushed through the system for ˜5 minutes before switching to a desired substrate solution. Once all the substrate solution had been added to the HPLC pump inlet reservoir, the reservoir was rinsed with ˜3×10 mL of the selected solvent to ensure that all of the substrate solution was flushed through the H-Cube®. Next, the run was stopped by using a H-Cube® interface and either new parameters were entered and a following run was started, or the H-Cube® was flushed with deoxygenated anhydrous ethanol and the H-Cube® and connected HPLC pump were shut down.

### Solid State NMR Acquisition

All ³¹P-NMR spectra were acquired with magic angle spinning (MAS) and ramped cross-polarization (RAMP-CP) on a Bruker Avance 500 NMR spectrometer, operating at 500.3 and 202.5 MHz for ¹H and ³¹P, respectively. The [Rh(NBD)((R)-5,5′-BINAP)](SbF₆) sample was packed into a 2.5 mm outer diameter rotor and spun at MAS frequencies 8 or 18 kHz; this sample was used to optimize the experimental conditions for the RAMP-CP experiments for all samples. The ¹H 90° pulse for the [Rh(NBD)((R)-5,5′-BINAP)](SbF₆) sample was 2.0 μs, the contact time was 3.0 ms, the acquisition time was 30 ms and the recycle delay was 3.0 s. All other ³¹P-NMR spectra were acquired on the same instrument, but were packed in 4.0 mm outer diameter NMR rotors. Samples for the latter were spun at 8.0 or 10.0 kHz, with a ¹H 90° pulse of 4.0 μs. AN other acquisition parameters were as outlined for the [Rh(NBD)((R)-5,5′-BINAP)](SbF₆) sample above.

### Neutron Activation Analysis Acquisition

Instrumental neutron activation analysis (NAA) was used to determine rhodium (Rh), barium (Ba), and antinomy (Sb) contents of used, and unused, catalyst samples. Samples (each weighing ≦55 mg) and standards were accurately weighed (or pipetted) into polyethylene micro-centrifuge tubes (˜175 μL volume), hermetically sealed and individually irradiated in the University of Alberta SLOWPOKE II nuclear reactor for 100 s at a nominal thermal neutron flux of 1×1011 n cm−2 s−1. Following a measured decay period (of between 20-30 s) the irradiated samples were individually counted for 100 s live-time at a sample-to-detector distance of 3 cm to measure the induced Rh gamma-ray activity. The Rh measurements were performed in open geometry using a 22% relative efficiency ORTEC hyperpure Ge detector (full-width at half maximum, FWHM, of 1.95 keV for the 1332.5 keV full energy peak of 60Co). The Ge detector was connected to a PC-based Aptec multichannel analyzer (MCA) card. Following a decay period of ˜4 h the samples were recounted for 1800 s to determine their Ba and Sb contents on the end-cap of an ORTEC high-purity FX-Profile Ge detector (Model GEM-FX8530P4) with a relative efficiency of 40% and a FWHM of 1.75 keV (for the 1332.5 keV 60Co photopeak) housed in a 10 cm Pb cave with Cu shield. FX Profile detector was coupled to an ORTEC DSPEC-Pro digital spectrometer. Elemental analysis was performed by the semi-absolute method of activation analysis for Rh and Ba. (Bergerioux, C.; Kennedy, G.; Zikovosky, L. J. Radioanal. Chem. 1979, 50, 22.) Antinomy was determined by absolute instrumental NAA. The nuclear reactions and relevant nuclear data for the quantification of the three elements measured are listed in the following table. A Sigma-Aldrich Fluka Analytical Rh AA standard solution (977.0 ug Rh/mL in 5% HCl) was used in quantifying Rh. Barium sulphate was used as comparator standard for the determination of the Ba. As noted above, Sb was determined by absolute (i.e., standard-less) NAA.

| | | |
|---|---|---|
| Nuclear Reaction | Half-life | Principal γ-ray(s) |

| | | | |
|---|---|---|---|
| ¹⁰³Rh (n, γ) ¹⁰⁴Rh | 42.3 | s | 555.8 keV |
| ¹³⁸Ba (n, γ) ¹³⁹Ba | 83.06 | m | 165.9 keV |

### Determination of Enantiomeric Excess

Products from catalytic hydrogenations were concentrated under reduced pressure and an aliquot was flushed through a Fluorosil™ plug using CH₂Cl₂ as an eluent to remove any catalyst residues. Retention times and chiral GC or HPLC conditions for the products are given below and the retention times were confirmed with racemic samples of the products. ¹H-NMR spectra recorded were identical to the authentic samples.

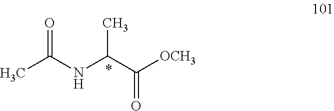

Enantiomeric excess of the product from hydrogenation of MAA (101) was determined through chiral GC, however the peaks did not fully separate on the column. The product was concentrated under reduced pressure and a solution was prepared in CH₂Cl₂ at a concentration of 2 mg/mL. Next, 1 μL was injected into the GC under the following conditions: helium carrier gas (20 psig); constant temperature of 80° C.; injector temperature of 220° C.; detector temperature of 220° C. Retention times for the two enantiomers were 75.7 min and 77.6 min.

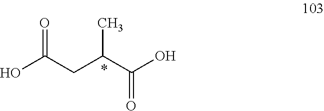

Enantiomeric excess of the product from hydrogenation of itaconic acid (103) was determined through chiral HPLC and confirmed with a racemic methylated compound (dimethyl methyl succinate, 104), which was obtained from Sigma-Aldrich. The product was first methylated by reaction with diazomethane. The methylated product was then concentrated under reduced pressure and a solution was prepared in THF at a concentration of 2 mg/mL. Next, 3 μL was injected into the HPLC under the following conditions: 30° C., 0.8 mL/min flow rate, mobile phase of 98:2 hexane:isopropanol. Retention times for the two enantiomers of the racemic methylated compound 104 were 7.6 min and 9.9 min. Methylated product from certain rhodium catalytic polymeric framework reactions only contained the enantiomer at 9.9 min. Therefore, ee was determined to be >99.9%.

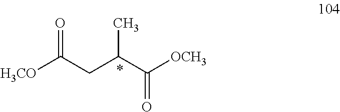

Enantiomeric excess of the product from hydrogenation of dimethyl itaconate (104) was determined through chiral HPLC and confirmed with the racemic compound, which was obtained from Sigma-Aldrich. Product was concentrated under reduced pressure and a solution was prepared in THF at a concentration of 2 mg/mL. Next, 3 μL was injected into the HPLC under the following conditions: 30° C., 0.8 mL/min flow rate, mobile phase of 98:2 hexane:isopropanol. Retention times for the two enantiomers were 7.5 min and 9.7 min.

### Example 1

### Hydrogenation of 3-buten-2-ol over catalytic polymeric framework 42 (poly-[Rh(NBD)((R)-5,5′-dinorimido-BINAP)](SbF₆)/BaSO₄)

The catalytic polymeric framework (CPF) 42 was chosen for initial experiments in the H-Cube® continuous-flow hydrogenation reactor because this catalyst does not require a silver salt to generate an active catalyst. The NBD ligand is removed by hydrogenation during the catalytic hydrogenation reaction, generating the active catalytic species [Rh((R)-5,5′-dinorimido-BINAP)]⁺. CPF 42 was first evaluated using 3-buten-2-ol (71) because it was found that 71 was a highly active substrate for allylic alcohol isomerizations. 71 was also known to undergo olefin hydrogenation and isomerization (Equation I), which allowed activity of the CPF to be evaluated for both hydrogenation and isomerization. The catalyst activation experiments using COF 42 in the H-Cube® are summarized in Table 2. To achieve 100% conversion, concentration of the substrate solution was diluted by a factor of three, to 0.077 M in THF, while other reaction conditions were kept constant.

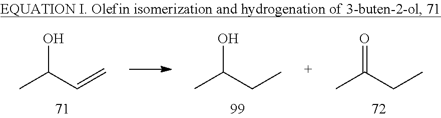

TABLE 2
Catalyst activation with 3-buten-2-ol^{a}

| | | | H₂ pressure | Conversion^{b} |
|---|---|---|---|---|
| Entry | Loading(Sub/Rh) | [Sub] | (bar) | (%) |
| 1 | 2000/1 | 0.23M | 30 | 54 |
| 2 | 2000/1 | 0.077M | 30 | 100 |
| 3 | 1000/1 | 0.077M | 30 | 100 |
| 4 | 1000/1 | 0.077M | 60 | 100 |
| 5 | 1000/1 | 0.077M | 0 | 0 |
| 6 | 20,000/1 | 0.077M | 30 | 100 |
| 7 | 1000/1 | 0.077M | 30 | 100 |

^{a}The reactions were carried out in THF at 50° C. with a flow rate of 0.8 mL/min. The same poly-[Rh(NBD)((R)-5.5′-dinorimido-BINAP)](SbF₆)/BaSO₄ CatCart ® (30 × 4 mm) was used for every entry.
^{b}Conversion was determined by ¹H-NMR and by comparison to authentic samples.

Once the catalyst was conditioned, the reaction was carried out under 60 bar (entry 4) and 0 bar (entry 5) to investigate the effect of hydrogen pressure on the ratio of isomerized product 72 to hydrogenated product 99. Increasing hydrogen pressure did not have any effect on the percent conversion (100%) or product distribution (7% isomerized product in both entries 3 and 4). There was 0% conversion for isomerization in the absence of hydrogen (entry 5); this suggests that when hydrogen is not present, the catalyst forms a relatively stable, catalytically inactive complex (or resting state). Under conditions of entry 6 and 7, it was demonstrated that a large turn over of 20 000 (100% conversion) could be achieved with a low catalyst loading (entry 6, with 100-200 times less catalyst than previously reported: Alamé, M.; Jahjah, M.; Pellet-Rostaing, S.; Lemaire, M.; Meille, V.; de Bellefon, C. J. Mol. Catal. A: Chem. 2007, 271, 18; Alamé, M.; Jahjah, M.; Berthod, M.; Lemaire, M.; Meille, V.; de Bellefon, C. J. Mol. Catal. A: Chem. 2007, 271, 205; Berthod, M.; Mignani, G.; Woodward, G.; Lemaire, M. Chem. Rev. 2005, 105, 1801; Rankic, D. A.; Hopkins, J. M.; Parvez, M.; Keay, B. A. Synlett. 2009, 15, 2513; Hopkins, J. M.; Dalrymple, S. A.; Parvez, M.; Keay, B. A. Org. Lett. 2005, 7, 3765; Cram, D. J.; Helgeson, R. C.; Peacock, S. C.; Kaplan, L. J.; Domeier, L. H.; Moreau, P.; Koga, K.; Mayer, J. M.; Chao, Y.; Siegel, M. G.; Hoffman, D. H.; Sogah, G. D. Y. J. Org. Chem. 1978, 43, 1930; Saluzzo, C.; Lemaire, M. Adv. Synth. Catal. 2002, 344, 915; Shimazu, S.; Ro, K.; Sento, T.; Ichikuni, N.; Uematsu, T. J. Mol. Catal. A: Chem. 1996, 107, 297; Guerreiro, P.; Ratovelomanana-Vidal, V.; Genet, J.-P.; Dellis, P. Tetrahedron Lett. 2001, 42, 3423; She, J.; Ye, L.; Zhu, J.; Yuan, Y. Catal. Lett. 2007, 116, 70; Bayardon, J.; Holz, J.; Schäffner, B.; Andrushko, V.; Verevkin, S.; Preetz, A.; Börner, A. Angew. Chem. Int. Ed. 2007, 46, 5971; Yinghuai, Z.; Carpenter, K.; Bun, C. C.; Bahnmueller, S.; Ke, C. P.; Srid, V. S.; Kee, L. W.; Hawthorne, M. F. Angew. Chem. Int. Ed. 2003, 42, 3792. (d) Altinel, H.; Avsar, G.; Yilmaz, M. K.; Guzel, B. J. Supercrit. Fluids 2009, 51, 202; Bainchini, C.; Barbaro, P.; Dal Santo, V.; Gobetto, R.; Meli, A.; Oberhauser, W.; Psaro, R.; Vizza, F. Adv. Synth. Catal. 2001, 343, 41; and McDonald, A. R.; Müller, C.; Vogt, D.; van Klink, G. P. M.; van Koten, G. Green Chem. 2008, 10, 424.), and that the catalyst remained active after such a large substrate loading run (entry 7).

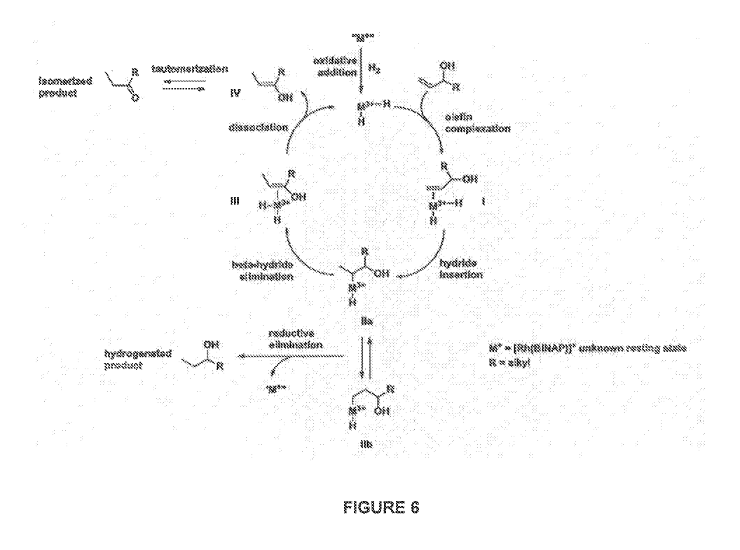

Without wishing to be bound by theory, a mechanism of hydrogenation and isomerization was proposed, which proceeds via metal hydride intermediates as shown in FIG. 6. Rh resting state complex (M+) undergoes oxidative addition with hydrogen followed by olefin complexation to form I. I then undergoes hydride insertion to form II, that can either reductively eliminate to produce the hydrogenated product or β-hydride eliminate to form III. Dissociation gives enol IV that can either tautomerize or re-enter the catalytic cycle to give the isomerized product. In the absence of hydrogen, neither the hydrogenated nor the isomerized product would be produced, which is consistent with results mentioned above.

### Example 2

### Secondary Allylic Alcohol Size Effects

In a previous study on isomerization of a series of allylic alcohols catalyzed by the CPF 42 (+AgSbF₆) (Corkum, E. G.; Kalapugama, S.; Hass, M. J.; Bergens, S. H. RSC Advances 2012, 2, 3473), it was shown that increasing chain length decreased rate of isomerization; secondary allylic alcohols containing alkyl chains with more than three carbons resulted in a decrease in catalytic activity. Activated CPF 42 was used for hydrogenation of a series of allylic alcohols to confirm/investigate the size effect. Substrates that were chosen for this study included 3-buten-2-ol (71), 1-penten-3-ol (73), 1-hexen-3-ol (74) and 1-hepten-3-ol (75), and the results are summarized in Table 3.

TABLE 3
Continuous-flow hydrogenation/isomerization of allylic alcohol substrates
catalyzed by rhodium catalyst-organic framework 42.^{a}

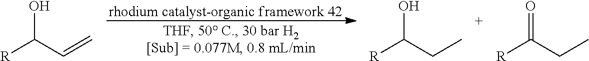

R = CH₃ (71), C₂H₅ (73), C₃H₇ (74), C₄H₉ (75)

| | | Total | | |
|---|---|---|---|---|
| | Loading | Conversion^{b} | Product Distribution^{b} (%) | |
| Sub | (Sub/Rh) | (%) | Hydrogenated | Isomerized |
| 71 | 2000/1 | 100 | 91 | 9 |
| 73 | 2000/1 | 100 | 75 | 25 |
| 74 | 2000/1 | 100 | 61 | 39 |
| 75 | 2000/1 | 70 | — | — |

^{a}Reactions were carried out in THF at 50° C. under 30 bar H₂ with a flow rate of 0.8 mL/min and substrate concentrations of 0.077M. Same poly-[Rh(NBD)((R)-5,5′-dinorimido-BINAP)](SbF₆)/BaSO₄ CatCart was u [symbol] sed for every entry.
^{b}Conversion and product distribution was determined by ¹H-NMR.

Substrates 71, 73 and 74, in the presence of CPF 42, were converted into a mixture of hydrogenated and isomerized with 100% conversion. Substrate 75 underwent 70% conversion. This is consistent with previous findings: the only substrate that was not fully converted into product had an alkyl chain longer than three carbons, suggesting that larger allylic alcohols can lead to a decrease in catalytic activity and rate of reaction. Substrates 71, 73 and 74 were all fully converted into product despite differences in alkyl chain length, suggesting that CPF 42 has a substrate size threshold that should not be exceeded for optimal catalytic activity.

### Example 3

### Hydrogenation of Dehydro Amino Acid Derivatives

In this example, rhodium catalytic polymeric framework 42 was used to catalyze continuous flow hydrogenation of α-acetamidocinnamic acid.

TABLE 4
Continuous-flow hydrogenation of α-acetamidocinnamic acid 100 catalyzed
by rhodium catalyst-organic framework 42.^{a}

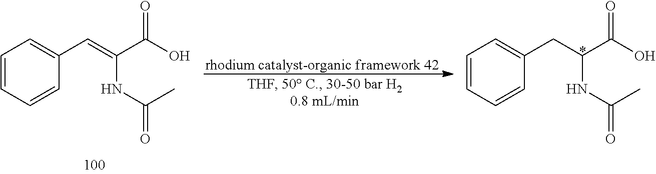

| | | H₂ Pressure | |
|---|---|---|---|
| Entry | Temp (° C.) | (bar) | Yieid^{b} (%) |
| 1 | 50 | 30 | 11 |
| 2 | 50 | 50 | 23 |

^{a}Reactions were carried out with 0.028M solutions of α-acetamidocinnamic acid in THF under the following conditions: Sub/Rh = 200/1, 0.8 mL/min flow rate. The same poly-[Rh(NBD)((R)-5,5′-dinorimido-BINAP)](SbF₆)/BaSO₄ CatCart was used for both entries.
^{b}Yield was determined by ¹H-NMR.

Referring to Table 4, yield was 11% (TON=22) under standard conditions (entry 1) and increased to only 23% (TON=46) under 50 atm of H₂ (entry 2). Without wishing to be bound by theory, it was postulated that the poor reactivity was due to a substrate size effect; specifically, the CPF 42-substrate size threshold was exceeded by the α-acetamidocinnamic acid substrate. Results obtained from hydrogenation of a smaller substrate, methyl 2-acetamido acrylate (MAA), by CPF 42 in the H-Cube® are summarized in Table 5.

TABLE 5
Continuous-flow hydrogenation of MAA catalyzed by rhodium
catalyst-organic framework 42.^{a}

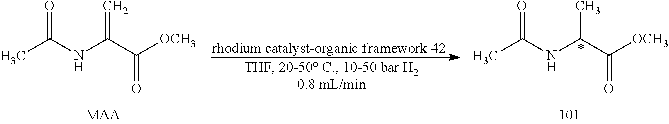

| | | H₂ Pressure | | |
|---|---|---|---|---|
| Entry | Temp (° C.) | (bar) | Yield^{b} (%) | ee^{c} (%) |
| 1 | 50 | 50 | 100 | 9.0 |
| 2 | 50 | 30 | 100 | 15.2 |
| 3 | 50 | 20 | 100 | 12.4 |
| 4 | 50 | 10 | 98 | 17.3 |
| 5 | 40 | 20 | 100 | 6.6 |
| 6 | 30 | 20 | 100 | 5.9 |
| 7 | 20 | 50 | 100 | 4.6 |
| 8 | 20 | 30 | 100 | 16.4 |

^{a}Reactions were carried out with 0.028M solutions of MAA in THF under the following conditions: Sub/Rh = 200/1, 0.8 mL/min flow rate.
^{b}Yield was determined by ¹H-NMR.
^{c}ee was determined by chiral GC.

Unlike 100, MAA was hydrogenated in 100% yield (TON=200) under standard conditions and 50 atm of H2 (entries 1 and 2). This result supports the hypothesis that substrate size threshold within CPF 42 was exceeded with α-acetamidocinnamic acid, 100. This finding is of particular importance as it demonstrates that CPF 42 can be used to selectively hydrogenate specific substrates within a given mixture based on substrate size exclusion.

Temperature and H₂ pressure were systematically varied to investigate the effect these parameters have on yield and ee. Changes in these reaction parameters had little or no effect on the overall yield, while ee generally increased with decreasing H₂ pressure (entries 1, 2 and 4) and with increasing temperature (entries 3, 5 and 6).

### Example 4

### Hydrogenation of Itaconic Acid

In this example, rhodium catalyst-organic framework 42 was used to catalyze the continuous flow hydrogenation of itaconic acid.

TABLE 6
Continuous-flow hydrogenation of itaconic acid 102 catalyzed by rhodium
catalyst-organic framework 42.^{a}

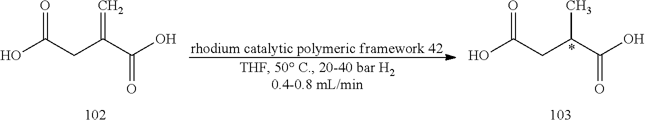

| | Flow Rate | H₂ Pressure | Yield^{d} (%) | |
|---|---|---|---|---|
| Entry | (mL/min) | (bar) | (TON) | ee^{e} (%) |
| 1 | 0.8 | 30 | 90 (180) | 21 |
| 2 | 0.8 | 40 | 81 (162) | — |
| 3^{b} | 0.6 | 30 | 92 (184) | 30 |
| 4^{b} | 0.4 | 20 | 93 (186) | — |
| 5^{a,c} | 0.8 | 30 | 98 (196) | — |

^{a}Reactions were carried out with 0.028M solutions of itaconic acid in THF under the following conditions: Sub/Rh = 200/1, 50° C. The same poly-[Rh(NBD)((R)-5,5′-dinorimido-BINAP)](SbF₆)/BaSO₄ CatCart was used for every entry.
^{b}The reactions were carried out with 0.014M solutions of itaconic acid in THF under the following conditions: Sub/Rh = 200:1, 50° C.
^{c}The substrate solution was run through the H-Cube twice.
^{d}Yield was determined by ¹H-NMR.
^{e}ee was determined by chiral HPLC.

In the first run (entry 1 of Table 6), under standard H-Cube® conditions (30 bar H₂, 50° C. and 0.8 mL/min flow rate), hydrogenated product 103 was obtained in 90% yield (TON=180). The yield actually dropped from 90% to 81% (TON=162) when the pressure was increased to 40 bar (entry 2). This suggests that the catalyst underwent some sort of decrease in activity from inhibition by itaconic acid. Reducing the flow rate and diluting the substrate concentration in half (entries 3 and 4) increased the yields to 92% (TON=184) and 93% (TON=186), respectively. Passing the reaction mixture twice through the H-Cube® (entry 5) resulted in a yield of 98% (TON=196).

Highest ee that was obtained for the hydrogenation was 30% (entry 3). Without wishing to be bound by theory, it was postulated that the lower enantioselectivity of the CPF 42 suggests an unfavorable substrate/framework or catalyst/framework interaction that was not present in the homogeneous systems, or that high H₂ pressures may be responsible. The high activity exhibited by the CPF did justify further substrate investigation.

### Example 5

### Hydrogenation of Dimethyl Itaconate

In this example, rhodium catalytic polymeric framework 42 was used to catalyze continuous flow hydrogenation of dimethyl itaconate to form 104.

TABLE 7
Continuous-flow hydrogenation of dimethyl itaconate 88 catalyzed by rhodium
catalyst-organic framework 42.^{a}

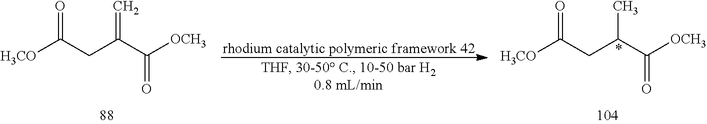

| | | H₂ Pressure | | |
|---|---|---|---|---|
| Entry | Temp (° C.) | (bar) | Yield^{c} (%) | ee^{d} (%) |
| 1 | 50 | 50 | 100 | 0.5 |
| 2 | 50 | 30 | 100 | 6.4 |
| 3 | 50 | 10 | 100 | 15.9 |
| 4 | 30 | 50 | 100 | 1.2 |
| 5 | 30 | 30 | 100 | 3.7 |
| 6 | 30 | 10 | 100 | 11.8 |
| 7^{b} | 50 | 30 | 72 | — |
| 8^{b} | 50 | 50 | 92 | — |

^{a}Reactions were carried out with 0.028M solutions of dimethyl itaconate in THF under the following conditions: Sub/Rh = 200/1, 0.8 mL/min flow rate. Same poly-[Rh(NBD)((R)-5,5′-dinorimido-BINAP)](SbF₆)/BaSO₄ CatCart was used for every entry.
^{b}Reactions were carried out with 0.077M solutions of dimethyl itaconate in THF under the following conditions: Sub/Rh = 10,000:1.
^{c}Yield was determined by ¹H-NMR.
^{d}ee was determined by chiral HPLC.

Changes in temperature (30-50° C.) and H₂ pressure (10-50 bar) had no effect on yield of hydrogenated product 104 (entries 1-6 of Table 7). However, enantioselectivity increased with decreasing H₂ pressure (entries 1-3, 4-6 of Table 7) and increased with increasing temperature (entries 3 and 6 of Table 7). Without wishing to be bound by theory, these trends suggest that optimal conditions for obtaining high enantioselectivities with this CPF 42 may involve use of low H₂ pressures and high temperatures.

Two large-scale runs were performed to test the catalyst's endurance. With a S/C ratio of 10,000:1, a TON of 7200 was achieved under the following conditions: 50° C., 30 bar of H2, 0.8 mL/min flow rate with a concentration of dimethyl itaconate of 0.077 M in THF (entry 7). In an attempt to increase the total percent yield, H₂ pressure was increased from 30 bar to 50 bar, causing a 20% increase in the yield, which corresponds to a total TON of 9200 (entry 8).

### Example 6

### Kinetic Resolution/Hydrogenation of α-Vinylbenzyl Alcohol

In this example, rhodium catalytic polymeric framework 42 was used to catalyze continuous flow hydrogenation of α-vinylbenzyl alcohol.

TABLE 8
Continuous-flow hydrogenation of α-vinylbenzyl alcohol 77 catalyzed by
rhodium catalyst-organic framework 42.^{a}

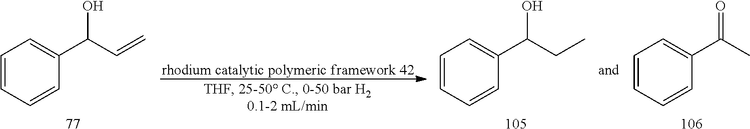

| | | Flow Rate | | H₂ Pressure | |
|---|---|---|---|---|---|
| Entry | [Sub] | (mL/min) | Temp (° C.) | (bar) | Conversion^{c} (%) |
| 1 | 0.028M | 0.8 | 50 | 50 | 100 |
| 2 | 0.028M | 0.8 | 50 | 30 | 100 |
| 3 | 0.028M | 0.8 | 50 | 10 | 100 |
| 4 | 0.077M | 1.2 | 50 | 10 | 100 |
| 5 | 0.077M | 1.6 | 50 | 10 | 100 |
| 6 | 0.077M | 1.6 | 25 | 10 | 100 |
| 7 | 0.077M | 2.0 | 25 | 10 | 100 |
| 8 | 0.1M | 2.0 | 25 | 10 | 100 |
| 9 | 0.1M | 2.0 | 25 | 1 | 100 |
| 10 | 0.1M | 2.0 | 25 | 0 | 0 |
| 11^{b} | 0.1M | 2.0 | 25 | 1 | 97 |

^{a}Reactions were carried out in THF. Same poly-[Rh(NBD)((R)-5,5′-dinorimido-BINAP)](SbF₆)/BaSO₄ CatCart was used for every entry.
^{b}This reaction was carried out in EtOH.
^{c}Conversion was determined by¹H-NMR and by comparison to authentic samples.

Substrate 77 was an active substrate, undergoing 100% conversion despite increasing concentration (0.028-0.1 M) and flow rate (0.8-2.0 mL/min) and decreasing temperature (25-50° C.) and H₂ pressure (1-50 bar) (entries 1-9). In typical asymmetric continuous-flow hydrogenation reactions, flow rates of <0.1 mL/min are necessary to ensure complete conversion (Shi, L.; Wang, X.; Sandoval, C. A.; Wang, Z.; Li, H.; Wu, J.; Yu, L.; Ding, K. Chem. Eur. J. 2009, 15, 9855; Balogh, S.; Farkas, G.; Madárdsz, J.; Szöllösy, A.; Kovács, J.; Darvas, F.; Urge, L.; Bakos, J. Green Chem. 2012, 14, 1146; and Augustine, R. L.; Tanielyan, S. K.; Mahata, N.; Gao, Y.; Zsigmond, A.; Yang, H. Appl. Catal., A. 2003, 256, 69). However, no kinetic resolution was observed.

Under 0 bar of H₂ pressure, there was no conversion of substrate 77 into either product 105 or 106 (entry 10). This result is in accordance with previous results and shows that the catalyst forms a relatively stable, catalytically inactive complex in the absence of hydrogen. This suggests that the catalyst can be stored in between catalytic runs without decomposing. CPF 42 also exhibited nearly the same activity in EtOH as in THF with only a slight decrease in percent conversion (entry 11, 97% conversion in EtOH and 100% conversion in THF). These results demonstrate the high activity, versatility and flexibility of the catalyst system.

### Example 7

### Utilization of the Poly-[RhCl((R)-5,5′-dinorimido-BINAP)]2/Ba-L-Tartrate catalytic polymeric framework (41) in the H-Cube®

Achiral support BaSO₄ was replaced by Ba-L-Tartrate and chloro-bridged dimeric CPF poly-[RhCl((R)-5,5′-dinorimido-BINAP)]2/Ba-L-Tartrate 41 was investigated to improve ee's of the above hydrogenations performed using the CPF 42. CPF 41 afforded excellent enantioselectivity in intramolecular cycloisomerizations of 1,6-enynes and exhibited excellent activity in isomerization of allylic alcohols. The CPF 41 required a silver salt to abstract the bridging chlorides to generate an active “[Rh((R)-5,5′-dinorimido-BINAP)]⁺” catalyst. The CatCart® was packed with both the CPF 41 and 25.5 equivalents of AgSbF₆ per rhodium center. 15.5 equivalents of AgSbF₆ were in the first layer of the CatCart®, followed by a mixture of 10 equivalents of AgSbF₆ and the rhodium CPF 41. It was expected that the solvent would dissolve AgSbF₆ at the start of the CatCart® and move it through the entire mixture of the rhodium catalytic polymeric framework. AgSbF₆ mixed throughout the CPF as expected to activate the more difficult to reach rhodium centers.

Similar to the previously studied CPF 42, the Ba-L-Tartrate supported CPF 41 was first tested in the hydrogenation of 3-buten-2-ol.

TABLE 9
Continuous-flow hydrogenation of 3-buten-2-ol (71) catalyzed
by rhodium catalytic polymeric framework 41.^{a}

| | Loading | H₂ pressure | Flow Rate | Conversion^{b} |
|---|---|---|---|---|
| Entry | (Sub/Rh) | (bar) | (mL/min) | (%) |
| 1 | 1000/1 | 30 | 0.8 | 95 |
| 2 | 1000/1 | 30 | 0.8 | 91 |
| 3 | 5000/1 | 40 | 0.8 | 93 |
| 4 | 5000/1 | 40 | 0.6 | 95 |

^{a}Reactions were carried out with 0.077M solutions 50° C. Same poly-[RhCl((R)-5,5′-dinorimido-BINAP)]₂/Ba-L-Tartrate CatCart was used for every entry.
^{b}Conversion was determined by ¹H-NMR and by comparison to authentic samples

Changing reaction conditions across entries 1-4 did not significantly change the % conversion (91% to 95%). These conversions were slightly lower than those observed for CPF 42. Difference in catalyst activity was attributed to the swellability of the CPFs.

TABLE 10
Continuous-flow hydrogenation of itaconic acid (102) catalyzed
by rhodium catalytic polymeric framework 41.^{a}

| | Loading | Flow Rate | H₂ Pressure | | |
|---|---|---|---|---|---|
| Entry | (Sub/Rh) | (mL/min) | (bar) | Yield^{c} (%) | ee^{d} (%) |
| 1^{b} | 200/1 | 0.6 | 30 | 62 | >99.9 |
| 2 | 100/1 | 0.4 | 30 | 78 | >99.9 |
| 3 | 100/1 | 0.4 | 50 | 91 | >99.9 |

^{a}These reactions were carried out with 0.0071M solutions of itaconic acid in THF at 50° C. Same poly-[RhCl((R)-5,5′-dinorimido-BINAP)]₂/Ba-L-Tartrate CatCart was used for every entry.
^{b}A 0.014M solution of itaconic acid in THF was used for this run.
^{c}Yield was determined by ¹H-NMR and by comparison to authentic samples.
^{d}ee was determined by chiral HPLC.

Itaconic acid (102) was chosen for study with this catalyst system as it provided the highest enantioselectivities from CPF 42. Overall, CPF 41 offered lower yields than CPF 42, but offered much higher enantioselectivities. Dilution of the substrate by half from 0.014 M to 0.0071 M, and lowering the flow rate from 0.6 ml/min to 0.4 mL/min (entry 2), increased the yield from 62% to 78%. Increasing H₂ pressure from 30 to 50 bar (entry 3) also increased the yield increased to 91%.

### Example 8

### Cartridge Lifetime Studies

Solid State NMR Results:

By comparing the solid state NMR spectra of oxidized 5,5′-dinorimido BINAP ligand with the [Rh(NBD)(N-BINAP)](SbF₆) monomer unit and the unused and used BaSO₄ supported poly-[Rh(NBD)(N-BINAP)](SbF₆), it was possible to detect an obvious presence of oxides in the used samples of supported catalyst (through the unisotropic distribution of the spinning side-bands). Without wishing to be bound by theory, it is possible that oxidation of the phosphines to phosphine oxides is responsible for deactivation of the first catalyst cartridge that was investigated.

As well, for the second catalyst cartridge (using the same supported supported poly-[Rh(NBD)(N-BINAP)](SbF₆)) a significant amount of phosphine oxides was observed to be present in the used sample. However, a slight chemical shift difference in this sample was seen as compared to the previous samples, which suggests that there may be two different phosphine environments present in the used catalyst sample. This could be due to the fact that COD was flushed through the catalyst, potentially creating a new phosphine environment.

Neutron Activation Analysis (NAA) Results:

By comparing the used and unused samples of BaSO₄ supported poly-[Rh(NBD)(N-BINAP)](SbF₆) and quantifying the amount of Rh in the samples with a Rh standard solution, it was possible to determine that 33% of the 0.00383 mg of Rh in the catalyst did manage to leach out of the support over the course of approximately a month. Thus, Rh leaching could potentially have resulted in deactivation of the catalyst. However, it was not clear whether Rh leaching occurred throughout the lifetime of the catalyst, or whether Rh leaching was due to low molecular weight polymers leaching from the bulk catalyst at the beginning of the catalyst lifetime.

Antimony levels in the used and unused samples were also analyzed and it was found that the antimony levels in the used sample had decreased by a factor of 10. This loss in antimony was attributed to the replacement of the SbF₆ counter-ion with deprotonated carboxylates, which could have come from any acidic substrate that was used (e.g., itaconic acid). Rh-carboxylates are well known and form relatively strong bonds, resulting in fewer Rh sites available to participate in catalysis, which could also explain loss of activity in the first catalyst cartridge.

CatCart® Lifetime Assessment:

A conclusion of the solid state NMR analysis was that it showed that the cause of catalyst deactivation was oxidation over the approximately one month of operation. Further, as the Neutron Activation Analysis shows, leaching of rhodium is not significant over the course of the one month of operation. Taken together, these results indicate that neither leaching nor intrinsic catalyst lifetime limits the lifetime of these cartridges. Rather, slow oxidation of the catalyst occurs over the month of operation when 4-5 litres of solvent are passed through the cartridge. It should be further noted that the second catalyst cartridge loaded with supported poly-[Rh(NBD)(N-BINAP)](SbF₆) was still 100% active after ˜55,700 turnovers. However, after encountering clogging problems with the H-Cube® and subsequent removal of the CatCart®) from the H-Cube® it was found that the rubber o-rings on the CatCart® had begun to degrade, likely due to the sheer volume of THF solvent that was passed through the system over the course of approximately one month, and was most likely responsible for the clogging issues experienced.

The data shows that the catalysts were killed; they did not die of old age. These results demonstrate the remarkably long lifetime of the supported catalysts described herein.

### Example 9

### Synthesis and Deposition of a Pd-Based Catalytic Polymeric Framework

The following example demonstrates the design, synthesis, and deposition of catalytic polymeric frameworks with various ligand systems and metal centers.

### Synthesis of [Pd((R,R)—NORPHOS)(η³-C₃H₅)]BF₄

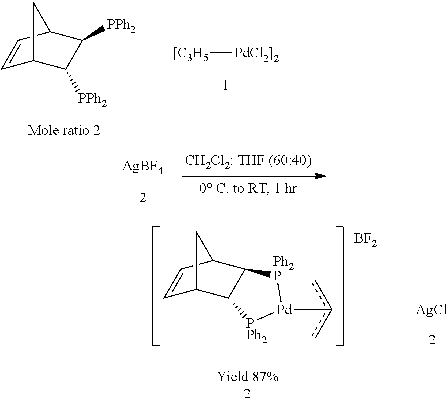

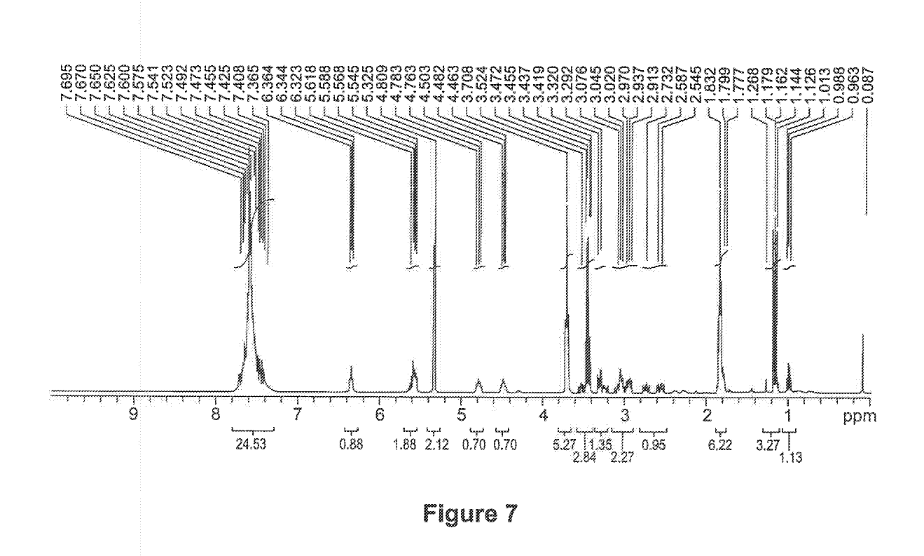

All solvents were distilled from an appropriate drying agent (CaH₂ for CH₂Cl₂, K/benzophenone for THF), under an atmosphere of nitrogen before use. All steps were carried out under nitrogen using standard schlenk techniques. A 50 mL side arm round bottom flask was charged with 41.90 mg of [(η³-C₃H₅)PdCl]₂ (1.08×10⁻⁴ mol), 100.00 mg of (R,R)—NORPHOS (2.16×10⁻⁴ mol), flushed with nitrogen gas, and sealed with a rubber septum. The round bottom flask's contents were dissolved in a CH₂Cl₂/THF solvent mixture (11.25 mL, 60:40 V/V), and stirred for 15 min at 0° C. In another 50 mL side arm flask, which was sealed with a rubber septum and flushed with nitrogen, 42.14 mg of AgBF₄ (2.16×10⁻⁴ mol) was dissolved in 7.5 mL THF in darkness (flask was wrapped in tin foil) and stirred at 0° C. for 15 min. The palladium-containing solution was transferred slowly, over 20 min via a cannula, into the flask containing the AgBF₄ solution. CH₂Cl₂ (11.25 mL) was used to rinse and fully transfer the Pd-containing solution. The combined solutions were stirred in darkness, for 15 min at 0° C., after addition was complete. Next, the reaction mixture was allowed to warm to room temperature slowly over 1 h, while stirring. The resulting pale-yellow solution was then filtered through a plug of Celite (5 g); the Celite plug and AgCl precipitate were washed with CH₂Cl₂ (2×5 mL). Solvent was removed from filtrate under reduced pressure to yield a yellow-brown powder (133 mg, yield 87%). ³¹P{¹H}NMR and ¹H NMR analysis was performed. 2R, 3R-NorPhos Ligand: ³¹P{¹H}NMR (CD₂Cl₂), δ 1.33, −3.22; ¹H NMR (CD₂Cl₂), δ 0.82 (1H), 1.032 (1H), 2.23 (1H), 2.77 (2H), 2.88 (1H), 6.02 (1H), 6.28 (1H), 7.34 (20H). [Pd((R,R)—NORPHOS)(η³-C₃H₅)]BF₄: ³¹P{¹H}NMR (CD₂Cl₂), δ 24.73, 25.04, 25.96, 26.00, 26.30, 26.61 9 (due to Pd—P coupling); the ¹H NMR spectrum is shown in FIG. 7.

ROMP Assembly of the Catalyst-Organic Framework.

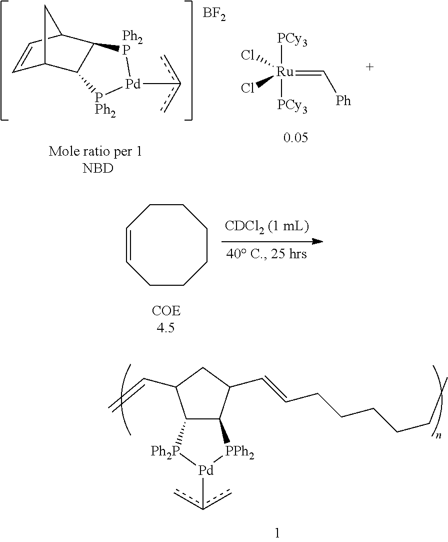

Under an atmosphere of nitrogen, 24.3 mg of [Pd((R,R)—NORPHOS)(η³-C₃H₅)]BF₄ (3.44×10⁻⁵ mol) was dissolved in 0.3 mL of CD₂Cl₂ in an NMR tube fitted with a rubber septum. Cyclooctene (COE) (14 μL, 1.03×10⁴ mol, distilled under nitrogen) was added with a gastight syringe. Grubb's catalyst (1^{st} Generation), trans-RuCl₂(═CHPh)(PCy₃)₂ (1.5 mg, 1.72×10⁻⁸ mol), was weighed in a glove box, transferred into an NMR tube with a septum, and dissolved in CD₂Cl₂ (0.3 mL) under a nitrogen atmosphere, resulting in a purple solution. Next, Grubb's solution was transferred, with a cannula, into the NMR tube containing the palladium complex and COE, and CD₂Cl₂ (0.4 mL) was used to rinse and fully transfer the Grubb's solution. The NMR tube's septum was sealed with paraffin tape, and the tube was placed in an oil bath heated to 40° C. After 24 h, a recorded ¹H NMR spectrum of the mixture showed that the COE was consumed, but only a small amount of the Pd complex had reacted. More cyclooctene (7 μL, 5.15×10⁻⁵ mol) was added to the mixture, and the mixture was heated for an additional 60 min at 40° C. A ¹H NMR spectrum of the subsequent mixture showed that ring-opening metathesis polymerization (ROMP) went to completion, and a catalyst-organic framework was made. This suggested that Grubb's 1^{st} Generation catalyst, trans-RuCl₂(=CHPh)(PCy₃)₂, converted into a more active form during the 24 h reaction period. Perhaps the catalysts PCy₃ ligands complexed, to some extent, to [Pd((R,R)—NORPHOS)(η³-C₃H₅)]BF₄, forming a more active form of the metathesis catalyst. ³¹P {¹H} NMR and ¹H NMR analysis was performed. Catalytic polymeric framework: ³¹P NMR (CD₂Cl₂), δ 28-34 (broad polymer peaks); ¹H NMR (CD₂Cl₂), δ 1.14-2.13 (poly alkyl, broad), 3.41-3.70 (norbornene protons under broad polymer peaks), 5.21-5.39 (polymer olefin region), 7.28-7.70 (polymer aryl+starting aryl overlap, broad).

Deposition of the Pd Catalytic Polymeric Framework onto BaSO₄

Clean BaSO₄ (1.84 g, washed with CH₂Cl₂, followed by diethyl ether and then dried under high vacuum overnight) was added to a round bottom flask equipped with a side arm (200 mL) and a magnetic stir bar. The flask containing BaSO₄ was placed under vacuum to dry for an additional 3 hrs. The flask was then backfilled with nitrogen. Next. CH₂Cl₂ (25.5 mL) was transferred with a cannula into the BaSO₄— containing flask and stirred to form a slurry. Then, a solution containing the catalyst-organic framework, made in the previous example (see Example XX), was transferred from its NMR tube to the BaSO₄/CH₂Cl₂ slurry with a cannula. CH₂Cl₂ (5 mL) was used to rinse and fully transfer the catalyst-organic framework solution. The polymer solution and BaSO₄ slurry were left to stir at room temperature for 1 h. Solvent was removed slowly under reduced pressure with rapid stirring to form a film of the catalyst-organic framework over the BaSO₄ support. The BaSO₄ deposited catalyst-organic framework was then washed with methanol (3×10 mL). Examination of the washings by ¹H and ³¹P NMR showed that all the palladium-polymer was deposited on the BaSO₄. The supported catalyst was obtained as an off-white powder in a 1.87 g yield.

### Example 10

### Functionalization of Ligands with Polymerizable Moieties

The following example demonstrates an ability to functionalize ligands with polymerizable moieties and/or pre-cursors to polymerizable moieties to facilitate their incorporation into a catalytic polymeric framework.

(S)-PhanePhos

### Synthesis of (S)-Phanephos oxide

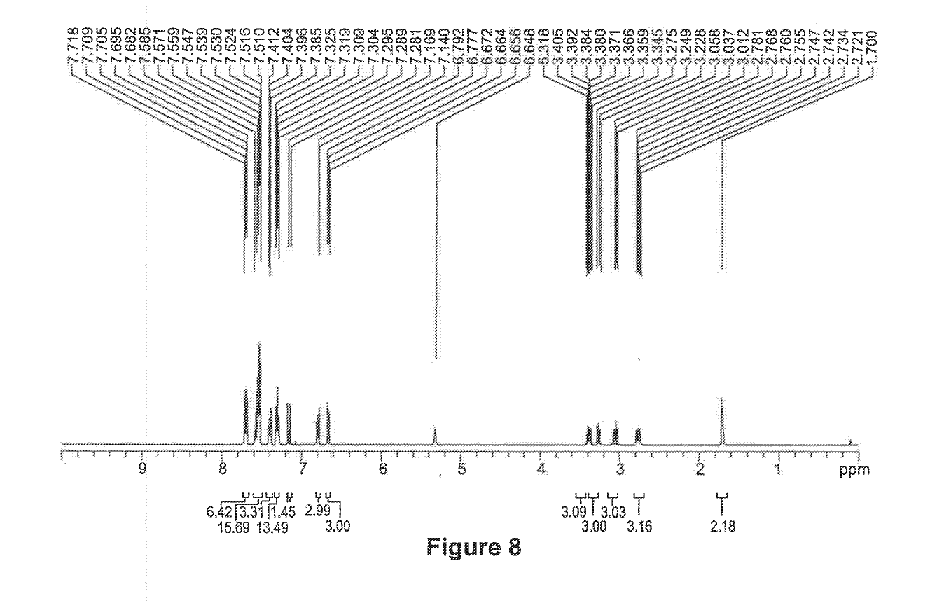

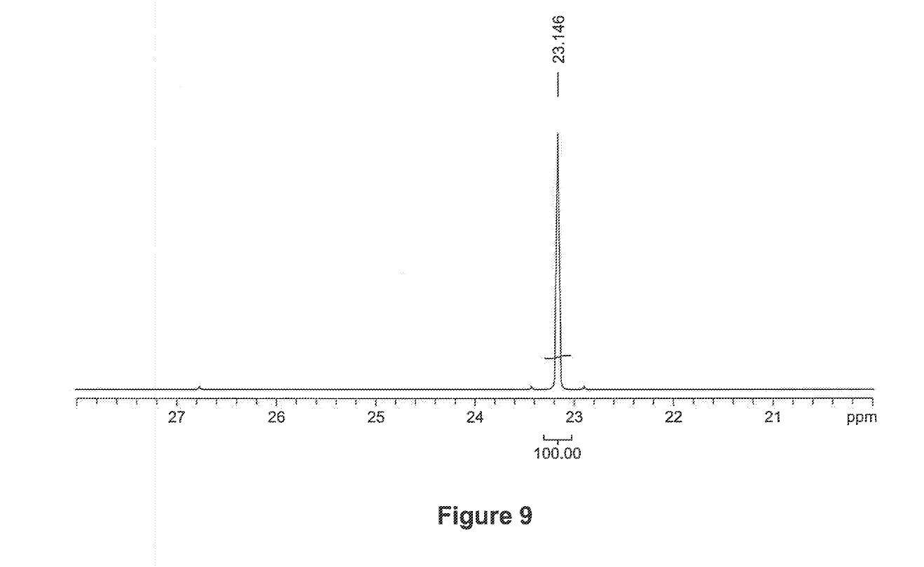

(S)-Phanephos (1.023 g, 1.73 mmol) was dissolved in dichloromethane (undistilled, 80 mL); 10% H₂O₂ (70 mL) was then added to said solution. Reaction mixture was stirred for 90 min, and then saturated Na₂S₂O₃ (˜200 mL) was added slowly to the reaction mixture until any excess H₂O₂ was neutralized. Using a separatory funnel (500 mL), the reaction mixture was washed with H₂O (3×60 mL) and saturated NaCl (3×60 mL). Organic layer was dried over anhydrous Na₂SO₄, filtered and concentrated under reduce pressure. A white solid was obtained as product (1.13 g, quantitative yield). ¹H NMR and ³¹P {¹H}NMR spectra were as shown in FIGS. 8 and 9, respectively.

Nitration of (S)-Phanephos oxide (Sub: HNO₃: HSO₄ ratio 1:2.1:1 at −28° C.).

(S)-Phanephos oxide (350 mg, 0.575 mmol) was weighted out into a 50 mL schlenk flask along with a % inch stir bar, and flushed with nitrogen gas for 10-15 min. ˜4.35 mL of 0.1323 M H₂SO₄/acetic anhydride standard solution (0.575 mmol, H₂SO₄) was added to the flask using a 10 mL syringe and stirred for 5-10 min until a clear solution was obtained. The above reaction mixture was then cooled to −28° C. for 20 min in an internal bath using a cooling circulating bath. 0.6908M HNO₃/acetic anhydride standard solution was also cooled to −28° C. In the same bath. ˜1.75 mL of HNO₃/acetic anhydride standard solution (1.207 mmol, HNO₃) was slowly added to the reaction flask using a cold 5 mL syringe, which was chilled in a freezer before use.

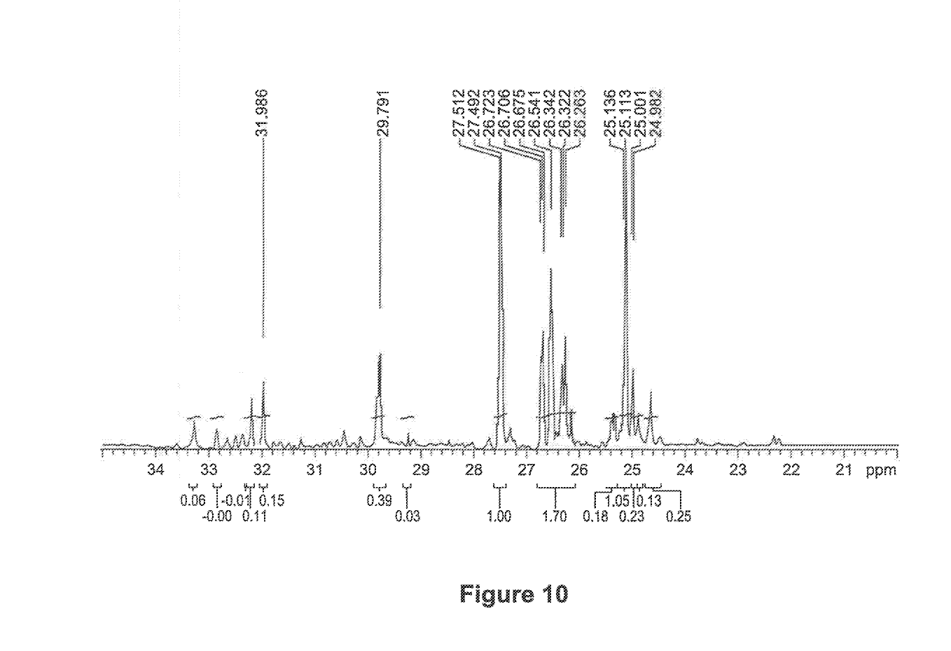

After 18 hrs, the reaction mixture was quenched by adding ice, followed by 20% NaOH until pH was basic. Flask was removed from the bath, and stirred for 2 min to ensure pH was still basic. It was then transferred to separatory funnel (1 L) and washed with 4×100 mL methylene chloride (undistilled), and the organic layer was collected into an Erlenmeyer flask. It was dried with anhydrous Na₂SO₄ and stirred for 60 min. The solution was gravity filtered and solvent was removed under reduced pressure to obtain a yellow, crude nitrated product (481 mg). ³¹P {¹H} NMR and ¹H NMR analysis was performed. ³¹P {¹H}NMR spectrum was as shown in FIG. 10.

Purification of Nitration Mixture by Flash Column Chromatography.

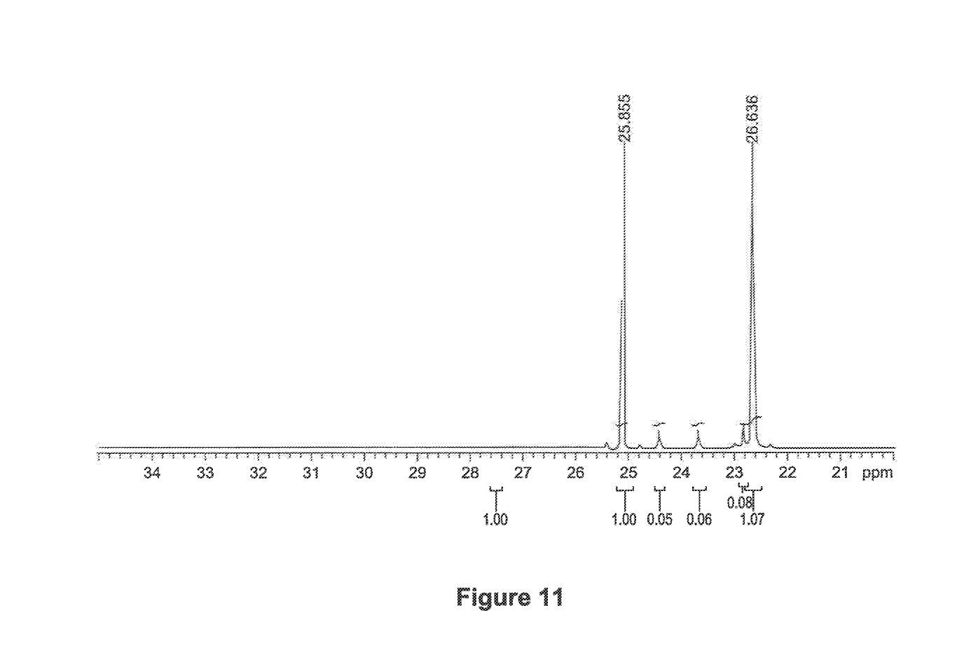

481 mg of nitrated product mixture (crude nitrated product from nitration reaction above) was purified through column chromatography (1:1 ethyl acetate/hexane, 26.5 g SiO₂). Flash chromatography largely separated one isomer of a mononitrated product in ˜29% isolated yield. Mass spectrometry analysis by ESI-TOF was found to be: C₄₀H₃₄NO₄P₂[M+H]⁺ m/z 654.1958 (calcd), 654.1947 (found). ³¹P {¹H}NMR spectrum was as shown in FIG. 11.

### 1,2-Bis[(R,R)-2,5-diphenylphospholano]ethane

### Synthesis of 1,2-Bis[(R,R)-2,5-diphenylphospholano]ethane[(R,R)-Ph-BPE]

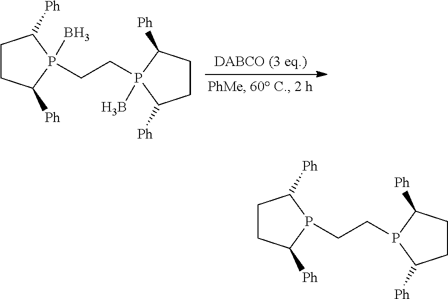

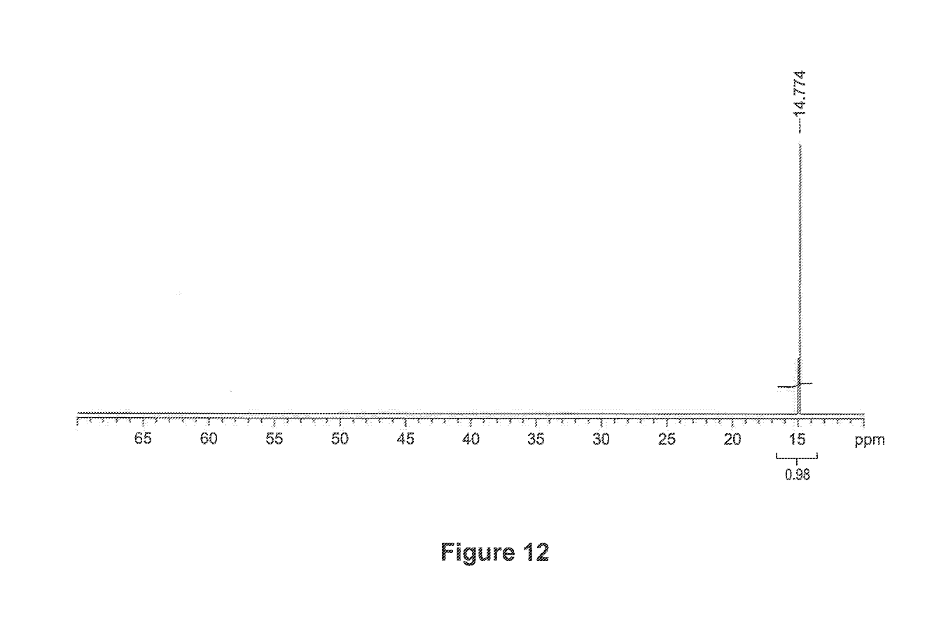

All solvents were distilled and degassed prior to use. 1,2-Bis[(R,R)-2,5-diphenylphospholano]ethane-borane adduct (1.60 g, 3.00 mmol) and DABCO® (1.01 g, 9.00 mmol) were charged to a 50 mL Schlenk flask inside a glove box. The flask was deoxygenated by evacuating and filling with nitrogen gas (×5). Distilled, degassed toluene (15 mL) was added, and the mixture was heated in an oil bath at 60° C. (external temperature) for 2 h. The reaction was allowed to cool to room temperature with stirring overnight. The solution was filtered through a pad of silica (10 g) under nitrogen, eluting with degassed toluene (30 mL). Isopropanol (10 mL) was added to the residue and the supernatant was removed by cannula transfer. The solid was washed with isopropanol (2×10 mL) and dried under vacuum to give the title compound (1.182 g, 78%). ³¹P{¹H}NMR and ¹H NMR analysis was performed. ³¹P{¹H}NMR spectrum was as shown in FIG. 12.

### Synthesis of (R,R)-Ph-BPE Oxide

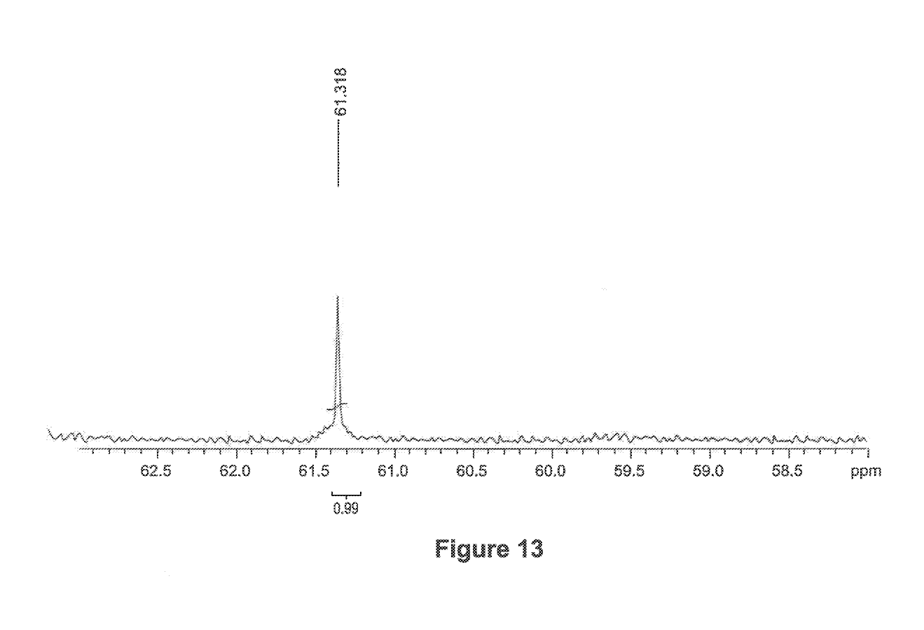

(R,R)-Ph-BPE Oxide (1.182 g, 2.33 mmol), as prepared above, was dissolved in −30 mL of methylene chloride (undistilled) and 10% H₂O₂ (135 mL, 396 mmol), and added directly in a round bottom flask with 1 inch stir bar. The reaction mixture was stirred for 1.5 h and kept in ice bath. It was quenched by slowly addling saturated Na₂S₂O₃. About ˜150 mL of Na₂S₂O₃ was added. Using a 500 mL separatory funnel, the reaction mixture was washed with H₂O (3×˜75 mL) and saturated NaCl solution (3×˜75 mL). The organic layer was dried over Na₂SO₄, gravity filtered, and concentrated under reduced pressure to afford a white powder of 1.44 g quantitatively. After putting the solid in high vacuum over night. ³¹P {¹H} NMR and ¹H NMR analysis was performed. ³¹P {¹H} NMR spectrum was as shown in FIG. 13.

### Nitration of (R,R)-Ph-BPE Oxide (sub:HNO₃:H₂SO₄ 2:1:5.7 at −18° C.)

(R,R)-Ph-BPE Oxide (473 mg, 0.878 mmol) was weighted out into a 100 mL schlenk flask along with a ½ inch stir bar, and flushed with nitrogen gas for 10-15 min. ˜35 mL of 0.1323M H₂SO₄/acetic anhydride standard solution (5.03 mmol, H₂SO₄) was added to the flask using a 10 mL syringe and stirred for 5-10 min. The obtained solution was turbid, and extra 3 mL of H₂SO₄/acetic anhydride standard solution was added in 1 mL portions until a clear solution was obtained. Said mixture was cooled to −18° C. for 20 min in an internal bath, using a cooling circulating bath. 0.6908M HNO₃/acetic anhydride standard solution was also cooled to −18° C. in the same bath. ˜2.5 mL of HNO₃/acetic anhydride standard solution was slowly added to the reaction flask using a cold 5 mL syringe that was chilled in a freezer before use.

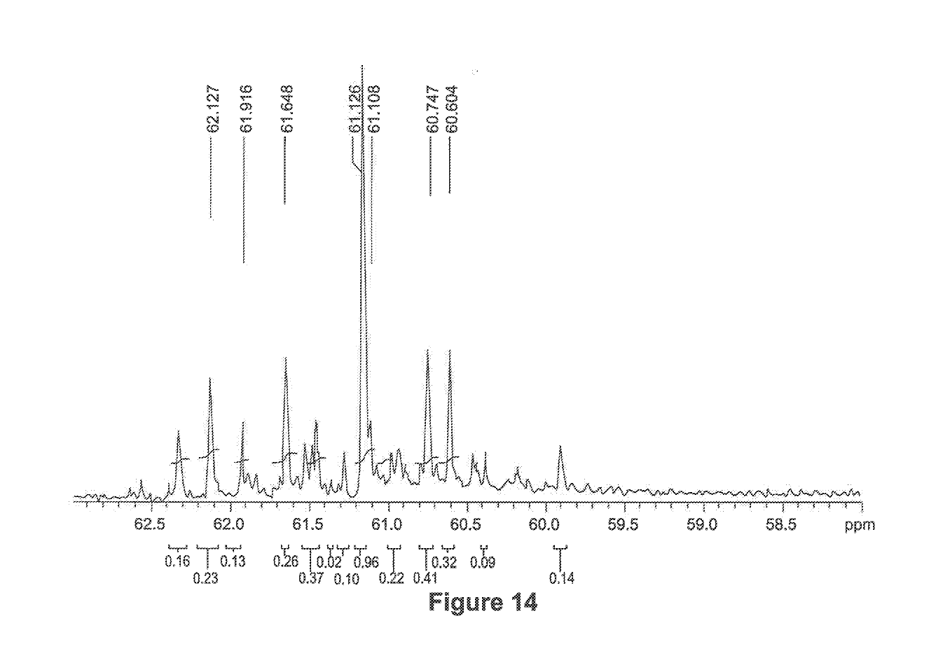

After 18 hrs, the reaction was quenched by adding ice, followed by 20% NaOH until pH was basic. The flask was removed from the bath, and stirred for 2 min to ensure pH was still basic. It was then transferred to a 1 L separatory funnel and washed with 4×100 mL of methylene chloride (undistilled), and the organic layer was collected into a 1 L Erlenmeyer flask. It was then dried with anhydrous Na₂SO₄ and stirred for 20 min. The solution was gravity filtered and solvent was removed under reduced pressure to obtain a yellow crude nitrated product (570 mg). ³¹P {¹H}NMR and ¹H NMR analysis was performed. ³¹P {¹H} NMR spectrum was as shown in FIG. 14.

### Purification of (R,R)-Ph-BPE Oxide Nitration Mixture by Flash Column Chromatography

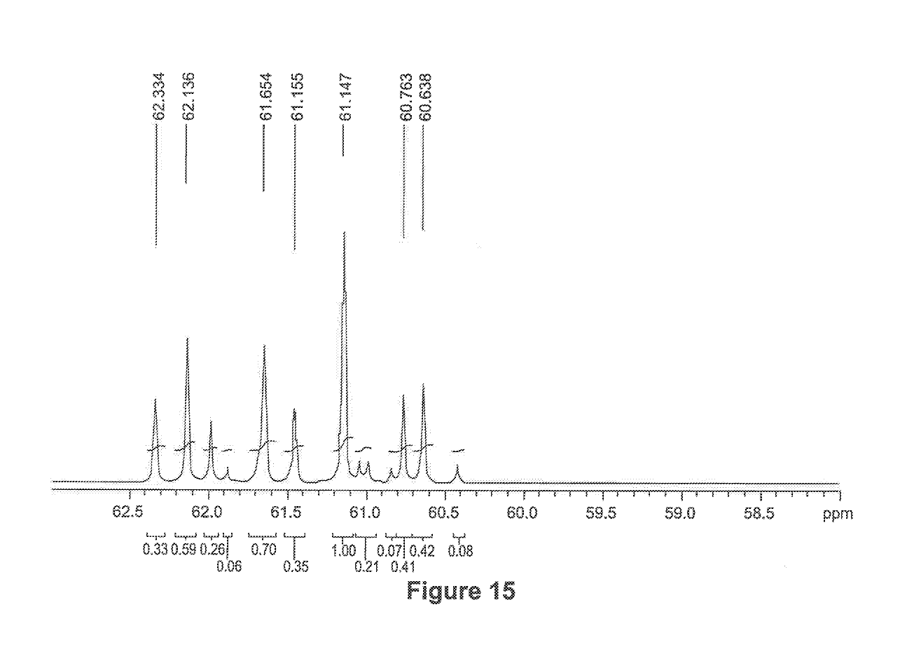

The nitration product from above reaction was purified through column chromatography. 100% ethyl acetate was used as eluent, with 25 g of silica used for first column to obtain a cleaner nitration mixture of 210 mg as indicated in the ³¹P {(H}NMR spectrum shown in FIG. 15.

It was then further purified via chromatography using a second column with 25 g of silica and 10%:90% ethanol:hexane. ³¹P {¹H}NMR, ¹H NMR and mass spectrometry analysis was performed on the isolated products, revealing: unreacted starting material (˜15%) a mixture of two mono-nitrated species (˜40% yield); and an ESI-TOF mass for C₃₄H₃₆NO₄P₂[M+H]⁺ m/z of 584.2114 (calcd), 584.2104 (found).

One of the mono-nitrated species was in largely pure form (˜25% yield), with an ESI-TOF mass for C₃₄H₃₆NO₄P₂[M+H]⁺ m/z of 584.2114 (calcd), 584.2111 (found). A symmetric di-nitrated species (˜15%) was also observed, with an ESI-TOF mass for C₃₄H₃₅N₂O₆P₂[M+H]⁺ m/z of 629.1965 (calcd), 629.1955 (found). Please note that the above reported yields are approximate, as they were determined from the second chromatography purification step that was carried out.

The resulting nitrated (S)-Phanephos and of (R,R)-Ph-BPE are suitable for use in reduction reactions to form the corresponding amines. As described in detail above, the amino compounds are then useful in the formation of catalyst-containing monomers for formation of a catalytic polymeric framework; by attachment of a suitable polymerizable moiety (e.g., norimido) via reaction at the added amino groups.

All publications, patents and patent applications mentioned in this Specification are indicative of the level of skill of those skilled in the art to which this invention pertains and are herein incorporated by reference to the same extent as if each individual publication, patent, or patent applications was specifically and individually indicated to be incorporated by reference.

The invention being thus described, it will be obvious that the same may be varied in many ways. Such variations are not to be regarded as a departure from the spirit and scope of the invention, and all such modifications as would be obvious to one skilled in the art are intended to be included within the scope of the following claims.

1-33. (canceled)

34. A system for use in a flow reactor, comprising:
a flow reactor cartridge comprising a catalytic polymeric framework covalently or non-covalently immobilized on and/or in a solid support material,
the catalytic polymeric framework being synthesized using an alternating ring-opening olefin metathesis polymerization (alt-ROMP) and comprising catalyst-containing monomer subunits, each separated by at least one non-catalyst-containing monomer subunit; and
the catalytic polymeric framework being derived from a transition metal catalyst;
with the proviso that the catalytic polymeric framework does not have the structure: 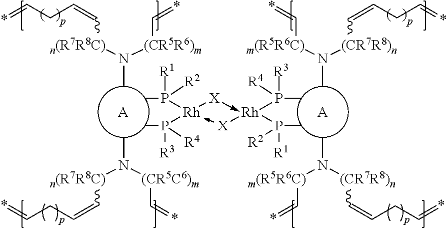
wherein
R¹, R², R³ and R⁴ are independently selected from phenyl and C₄₋₈cycloalkyl, the latter two groups being unsubstituted or substituted, where possible, with 1, 2, 3, 4, or 5 groups independently selected from C₁₋₆alkyl, OC₁₋₆alkyl and halo; [symbol]
s a binaphthyl group or a derivative of a binaphthyl group, each being unsubstituted or substituted with one or more groups independently selected from C₁₋₆alkyl, OC₁₋₆alkyl and halo;
R⁵, R⁶, R⁷ and R⁸ are independently selected from H, C₁₋₆alkyl, OC₁₋₆alkyl and halo; or R⁵ and R⁶ and/or R⁷ and R⁸ are ═O; or one of R⁵ and R⁶ is linked to one of R⁷ and R⁸ to form, together with the atoms to which they are attached and the atoms connecting them, a monocyclic, bicyclic or tricylic ring system; R⁵, R⁶, R⁷ and R⁸ in each methylene unit is the same or different, and [symbol]
means the double bond attached to this bond is in the cis or trans configuration, if applicable;
m and n are, independently, an integer between and including 0 and 10; p is an integer between and including 1 and 14; and
X is an anionic ligand.

35. The system of claim 34, wherein the catalyst-containing monomer subunits comprise a diphosphine ligand.

36. The system of claim 35, wherein each catalyst-containing monomer subunit is derived from a monomer having the structure: 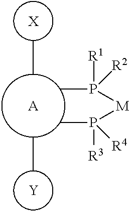
wherein
A is a substituted or unsubstituted aliphatic or aryl group;
X and Y are each independently a polymerizable moiety, wherein one of X or Y may be absent;
R¹, R², R³ and R⁴ are independently selected from aryl (e.g., phenyl), and C₄₋₈ cycloalkyl, the latter two groups being unsubstituted or substituted, where possible, with 1, 2, 3, 4, or 5 groups independently selected from C₁₋₆alkyl, OC₁₋₆alkyl and halo, or R¹ and R² and/or R³ and R⁴ together with the atoms to which they are attached form a substituted or unsubstituted cycloalkyl; and
M is a transition metal, optionally bound to another ligand or combination of ligands.

37. The system of claim 36, wherein the polymerizable moiety is selected from the group consisting of:

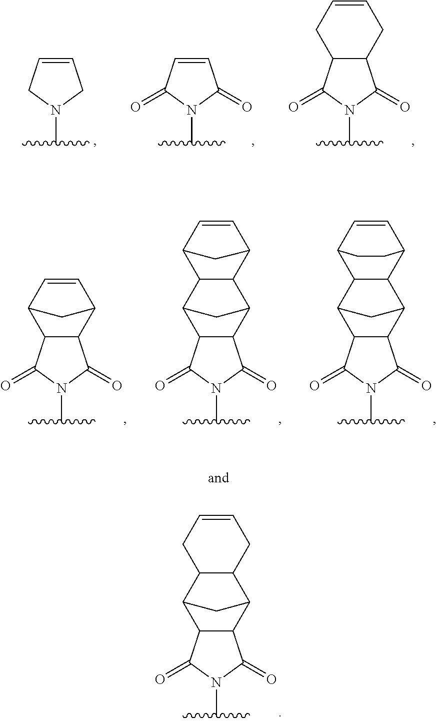

38. The system of claim 34, wherein the catalyst-containing monomer subunit is derived from a catalyst comprising a ligand that is

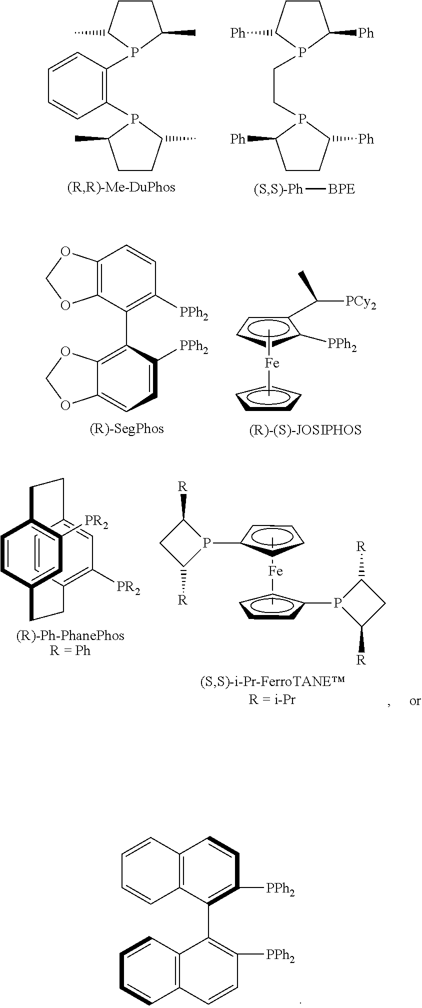

39. The system of claim 34, wherein the catalyst-containing monomer subunit comprises 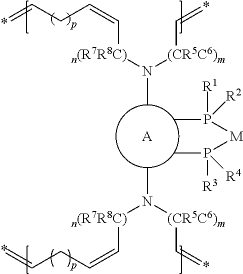
wherein
R¹, R², R³ and R⁴ are independently selected from aryl and C₄₋₈cycloalkyl, the latter two groups being unsubstituted or substituted, where possible, with 1, 2, 3, 4, or 5 groups independently selected from C₁₋₆alkyl, OC₁₋₆alkyl and halo;
A is a binaphthyl group or a derivative of a binaphthyl group, each being unsubstituted or substituted with one or more groups independently selected from C₁₋₆alkyl, OC₁₋₆alkyl and halo;
R⁵, R⁶, R⁷ and R⁸ are independently selected from H, C₁₋₆alkyl, OC₁₋₆alkyl and halo; or R⁵ and R⁶ and/or R⁷ and R⁸ are ═O; or one of R⁵ and R⁶ is linked to one of R⁷ and R⁸ to form, together with the atoms to which they are attached and the atoms connecting them, a monocyclic, bicyclic or tricylic ring system; R⁵, R⁶, R⁷ and R⁸ in each methylene unit is the same or different, and means the double bond attached to this bond is in the cis or trans configuration, if applicable;
m and n are, independently, an integer between and including 0 and 10;
p is an integer between and including 1 and 14; and
M is the transition metal, optionally bound to another ligand or combination of ligands.

40. The system of claim 39, wherein A is a binaphthyl group, or a derivative of a binaphthyl group, each being unsubstituted or substituted with one or more groups independently selected from C₁₋₆alkyl, OC₁₋₆alkyl and halo.

41. The system of claim 39, wherein R⁵, R⁶, R⁷ and R⁸, m and n, together with the atoms to which they are attached and the atoms connecting them, form a group selected from:

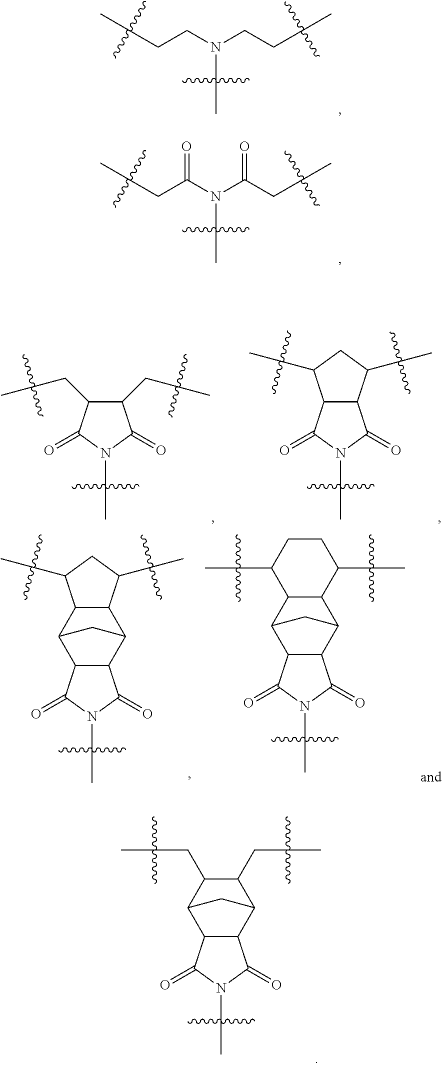

42. The system of any one of claim 34, wherein the transition metal is Cr, Mo, Fe, Ru, Os, Co, Rh, Ir, Ni, Pd, Pt, Cu, Ag, and/or Au.

43. The system of claim 39, wherein the catalyst-containing monomer subunit comprises

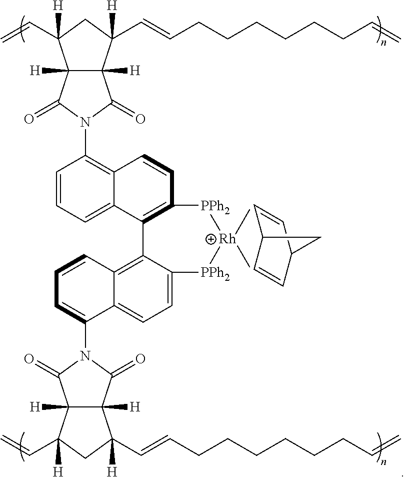

44. The system claim 34, wherein the solid support material comprises BaSO₄, barium (L)- and (D)-tartrates, aluminum oxide (Al₂O₃), silica (SiO₂), Fe₃O₄, Teflon™, Celite™, AgCl, sand, or any combination thereof.

45. The system of claim 34, wherein the flow reactor is a continuous flow reactor, such as an H-Cube® reactor.

46. The system of claim 34, which additionally comprises means for generating active catalyst.

47. The system of claim 46, wherein the means for generating active catalyst comprises a silver salt, such as AgSbF₆.

48. A method for metal-catalyzed organic synthesis comprising flowing a substrate for an organic synthesis through a flow reactor system of claim 34; and, optionally, isolating one or more products of the organic synthesis from the flow reactor system.

49. The method of claim 48, wherein the organic synthesis is any reaction that benefits from the presence or use of a metal catalyst, such as cycloisomerization, hydrosilation, hydrogenation, conjugate addition, or cross-coupling.

50. The method of claim 49, wherein the hydrogenation is an ester hydrogenation, an amide hydrogenation or a ketone hydrogenation.

51. The method of claim 49, wherein the organic synthesis is an asymmetric synthesis that affords an asymmetric or chiral product.

52. A composite material comprising:
(i) a catalytic polymeric framework synthesized using an alternating ring-opening olefin metathesis polymerization (alt-ROMP), comprising catalyst-containing monomer subunits, each separated by at least one non-catalyst-containing monomer subunit, and
(ii) a solid support material;
the catalytic polymeric framework being covalently or non-covalently immobilized on and/or in said support material, and
the catalytic polymeric framework being optionally derived from a transition metal catalyst;
wherein, when the catalytic polymeric framework is derived from a transition metal catalyst, the catalytic polymeric framework does not have the structure: 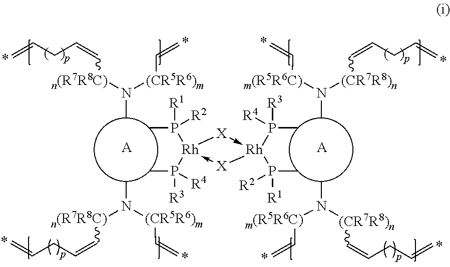
wherein
R¹, R², R³ and R⁴ are independently selected from phenyl and C₄₋₈cycloalkyl, the latter two groups being unsubstituted or substituted, where possible, with 1, 2, 3, 4, or 5 groups independently selected from C₁₋₆alkyl, OC₁₋₆alkyl and halo; [symbol]
s a binaphthyl group or a derivative of a binaphthyl group, each being unsubstituted or substituted with one or more groups independently selected from C₁₋₆alkyl, OC₁₋₆ alkyl and halo;
R⁵, R⁶, R⁷ and R⁸ are independently selected from H, C₁₋₆alkyl, OC₁₋₆ alkyl and halo; or R⁵ and R⁶ and/or R⁷ and R⁸ are ═O; or one of R⁵ and R⁶ is linked to one of R⁷ and R⁸ to form, together with the atoms to which they are attached and the atoms connecting them, a monocyclic, bicyclic or tricylic ring system; R⁵, R⁶, R⁷ and R⁸ in each methylene unit is the same or different, and [symbol]
means the double bond attached to this bond is in the cis or trans configuration, if applicable;
m and n are, independently, an integer between and including 0 and 10; p is an integer between and including 1 and 14; and
X is an anionic ligand; 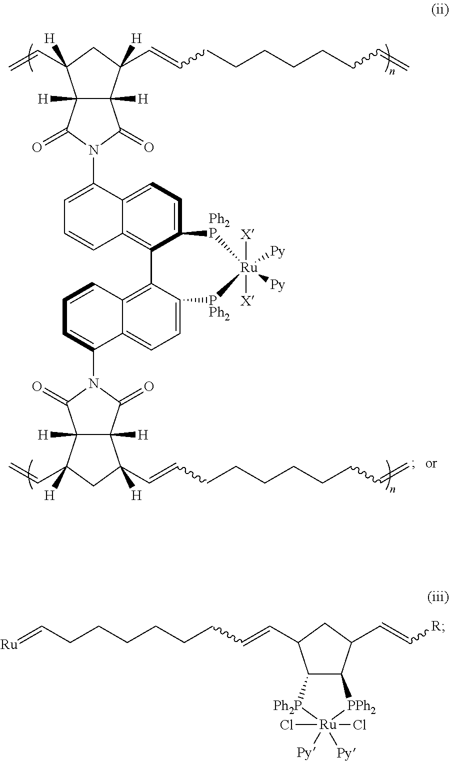
wherein,
when the structure is (ii), each Py is an unsubstituted pyridine ring, or together they are ((1R, 2R)-1,2-diphenylethylenediamine, or (R)-1,1-bis(4-methoxyphenyl)-3-methyl-1,2-butanediamine; and each X′ is Cl, or, when both Py together are ((1R, 2R)-1,2-diphenylethylenediamine, each X′ is H, Cl, or one X′ is H and the other X′ is OⁱPr; and
when the structure is (iii), R is an unsubstituted phenyl group, and each Py′ is an unsubstituted pyridine ring, or together they are ((1R, 2R)-1,2-diphenylethylenediamine.

53. The composite material of claim 52, wherein each catalyst-containing monomer subunit comprises a diphosphine ligand.

54. The composite material of claim 53, wherein each catalyst-containing monomer subunit is derived from a monomer having the structure: 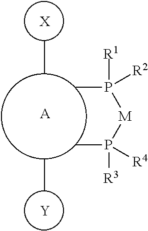
wherein
A is a substituted or unsubstituted aliphatic or aryl group;
X and Y are each independently a polymerizable moiety, wherein one of X or Y may be absent;
R¹, R², R³ and R⁴ are independently selected from aryl (e.g., phenyl), and C₄₋₈cycloalkyl, the latter two groups being unsubstituted or substituted, where possible, with 1, 2, 3, 4, or 5 groups independently selected from C₁₋₆alkyl, OC₁₋₆ alkyl and halo, or R¹ and R² and/or R³ and R⁴ together with the atoms to which they are attached form a substituted or unsubstituted cycloalkyl; and
M is a transition metal, optionally bound to another ligand or combination of ligands.

55. The composite material of claim 54, wherein the polymerizable moiety is selected from the group consisting of:

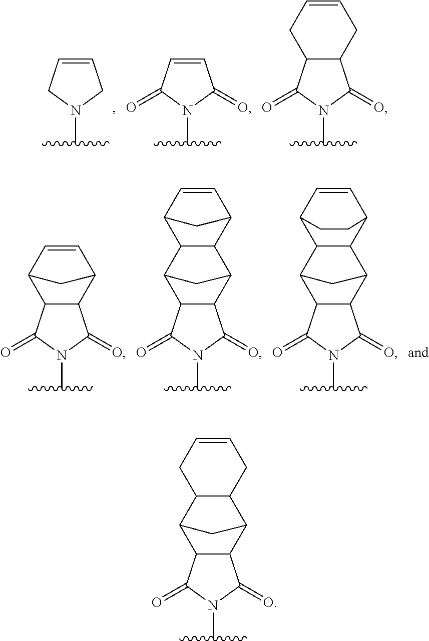

56. The composite material of claim 53, wherein the catalyst-containing monomer subunit is derived from a catalyst comprising a ligand that is

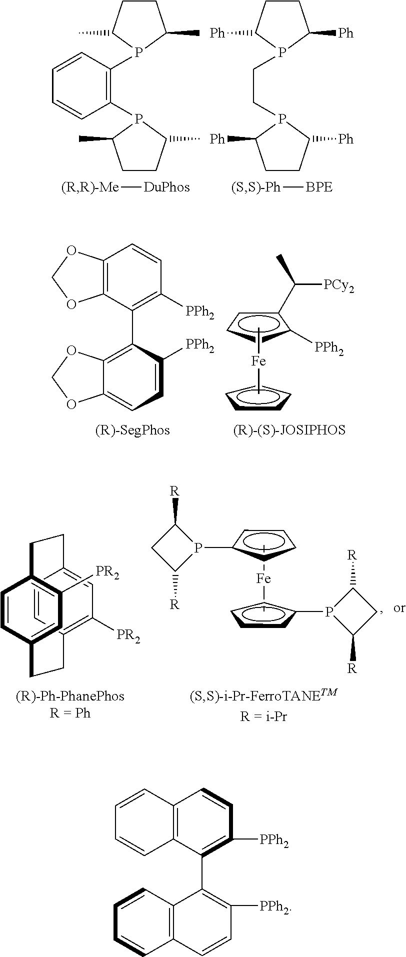

57. The composite material of claim 53, wherein the catalyst-containing monomer subunit comprises 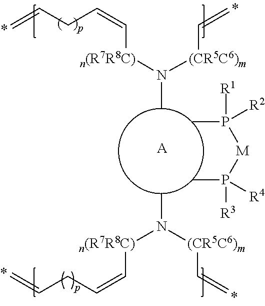
wherein
R¹, R², R³ and R⁴ are independently selected from aryl and C₄₋₈cycloalkyl, the latter two groups being unsubstituted or substituted, where possible, with 1, 2, 3, 4, or 5 groups independently selected from C₁₋₆alkyl, OC₁₋₆alkyl and halo;
A is a binaphthyl group or a derivative of a binaphthyl group, each being unsubstituted or substituted with one or more groups independently selected from C₁₋₆alkyl, OC₁₋₆alkyl and halo;
R⁵, R⁶, R⁷ and R⁸ are independently selected from H, C₁₋₆alkyl, OC₁₋₆alkyl and halo; or R⁵ and R⁶ and/or R⁷ and R⁸ are ═O; or one of R⁵ and R⁶ is linked to one of R⁷ and R⁸ to form, together with the atoms to which they are attached and the atoms connecting them, a monocyclic, bicyclic or tricylic ring system; R⁵, R⁶, R⁷ and R⁸ in each methylene unit is the same or different, and means the double bond attached to this bond is in the cis or trans configuration, if applicable;
m and n are, independently, an integer between and including 0 and 10;
p is an integer between and including 1 and 14; and
M is the transition metal, optionally bound to another ligand or combination of ligands.

58. The composite material of claim 57, wherein A is a binaphthyl group, or a derivative of a binaphthyl group, each being unsubstituted or substituted with one or more groups independently selected from C₁₋₆alkyl, OC₁₋₆alkyl and halo.

59. The composite material of claim 57, wherein R⁵, R⁶, R⁷ and R⁸, m and n, together with the atoms to which they are attached and the atoms connecting them, form a group selected from:

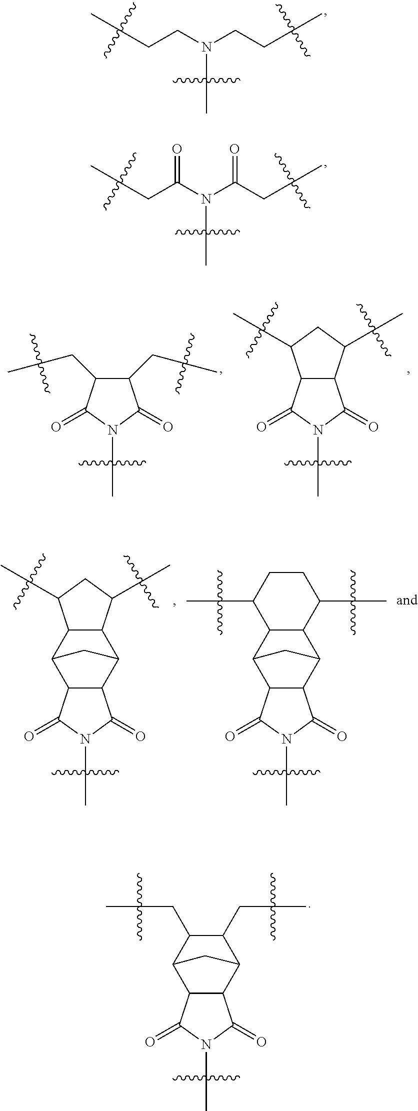

60. The composite material of claim 53, wherein the transition metal is Cr, Mo, Fe, Ru, Os, Co, Rh, Ir, Ni, Pd, Pt, Cu, Ag, and/or Au.

61. The composite material of claim 57, wherein the catalyst-containing monomer subunit comprises

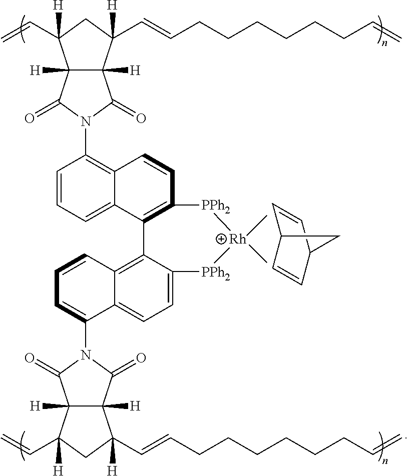

62. The composite material of claim 52, wherein the solid support material comprises BaSO₄, barium (L)- and (D)-tartrates, aluminum oxide (Al₂O₃), silica (SiO₂), Fe₃O₄, Teflon™, Celite™, AgCl, sand or any combination thereof.

63. The composite material of claim 52, for use in a flow reactor system, such as in a flow reactor cartridge.

64. The composite material of claim 63, wherein the flow reactor system is a continuous flow reactor, such as an H-Cube® reactor.

65. The composite material of claim 52, which additionally comprises means for generating active catalyst.

66. The composite material of claim 65, wherein the means for generating active catalyst comprises a silver salt, such as AgSbF₆.

67. A method for metal-catalyzed organic synthesis comprising flowing a substrate for an organic synthesis through a flow reactor system comprising the composite material of claim 53; and, optionally, isolating one or more products of the organic synthesis from the flow reactor system.

68. The method of claim 67, wherein the organic synthesis is any reaction that benefits from the presence or use of a metal catalyst, such as cycloisomerization, hydrosilation, hydrogenation, conjugate addition, or cross-coupling.

69. The method of claim 68, wherein the hydrogenation is an ester hydrogenation, an amide hydrogenation or a ketone hydrogenation.

70. The method of claim 68, wherein the organic synthesis is an asymmetric synthesis that affords an asymmetric or chiral product.

71. A method of preparing the immobilized catalytic polymeric framework of the flow reactor system of claim 34, said method comprising the steps of:
(a) derivatizing a catalyst to add one or more polymerizable moieties to a ligand of the catalyst to form a catalyst-containing monomer;
(b) polymerizing the catalyst-containing monomer with a non-catalyst-containing monomer using alternating ring-opening metathesis polymerization (alt-ROMP) to form the catalytic polymeric framework; and
(c) contacting the catalytic polymeric framework with a solid support material under conditions suitable for immobilization of the catalytic polymeric framework on and/or in the support material.

72. A method of preparing a catalytic polymeric framework, said method comprising the steps of:
(a) derivatizing a catalyst to add one or more polymerizable moieties to a ligand of the catalyst to form a catalyst-containing monomer;
(b) polymerizing the catalyst-containing monomer with a non-catalyst-containing monomer using alternating ring-opening metathesis polymerization (alt-ROMP) to form the catalytic polymeric framework;
wherein the catalytic polymeric framework does not have the structure: 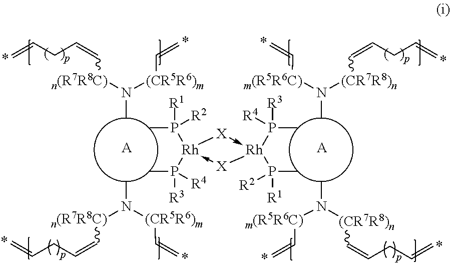
wherein
R¹, R², R³ and R⁴ are independently selected from phenyl and C₄₋₈cycloalkyl, the latter two groups being unsubstituted or substituted, where possible, with 1, 2, 3, 4, or 5 groups independently selected from C₁₋₆alkyl, OC₁₋₆alkyl and halo; [symbol] is a binaphthyl group or a derivative of a binaphthyl group, each being unsubstituted or substituted with one or more groups independently selected from C₁₋₆alkyl, OC₁₋₆alkyl and halo;
R⁵, R⁶, R⁷ and R⁸ are independently selected from H, C₁₋₆alkyl, OC₁₋₆alkyl and halo; or R⁵ and R⁶ and/or R⁷ and R⁸ are ═O; or one of R⁵ and R⁶ is linked to one of R⁷ and R⁸ to form, together with the atoms to which they are attached and the atoms connecting them, a monocyclic, bicyclic or tricylic ring system; R⁵, R⁶, R⁷ and R⁸ in each methylene unit is the same or different, and [symbol]
means the double bond attached to this bond is in the cis or trans configuration, if applicable;
m and n are, independently, an integer between and including 0 and 10; p is an integer between and including 1 and 14; and
X is an anionic ligand; 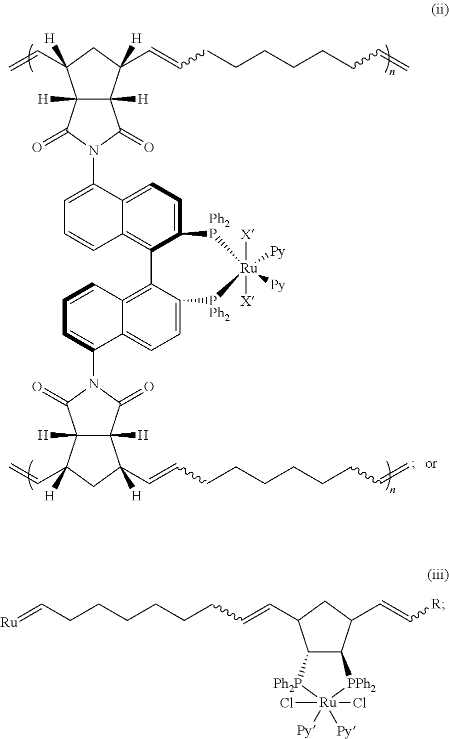
wherein,
when the structure is (ii), each Py is an unsubstituted pyridine ring, or together they are ((1R, 2R)-1,2-diphenylethylenediamine, or (R)-1,1-bis(4-methoxyphenyl)-3-methyl-1,2-butanediamine; and each X′ is Cl, or, when both Py together are ((1R, 2R)-1,2-diphenylethylenediamine, each X′ is H, Cl, or one X′ is H and the other X′ is OⁱPr; and
when the structure is (iii), R is an unsubstituted phenyl group, and each Py′ is an unsubstituted pyridine ring, or together they are ((1R, 2R)-1,2-diphenylethylenediamine.

73. The method of claim 72, wherein the catalyst is a transition metal catalyst.

74. The method of claim 72, wherein the catalyst comprises a diphosphine ligand.

75. The method of claim 74, wherein the catalyst comprises a ligand that is

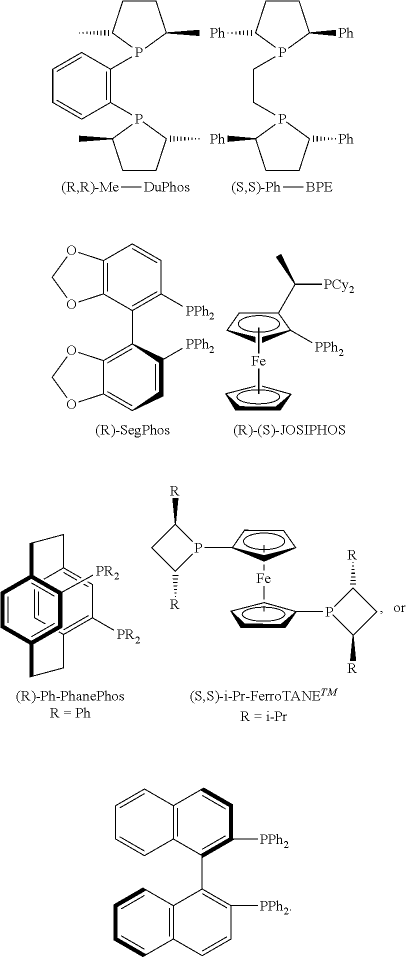

76. The method of claim 72, wherein the transition metal is Ru, Rh, Pd, Pt, Ir, Fe, Ni or Co.

77. A catalytic polymeric framework prepared by the method of claim 72.

78. A catalyst-containing monomer having the structure: 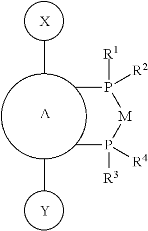
wherein
A is a substituted or unsubstituted aliphatic or aryl group;
X and Y are each independently a polymerizable moiety, wherein one of X or Y may be absent;
R¹, R², R³ and R⁴ are independently selected from aryl (e.g., phenyl), and C₄₋₈cycloalkyl, the latter two groups being unsubstituted or substituted, where possible, with 1, 2, 3, 4, or 5 groups independently selected from C₁₋₆alkyl, OC₁₋₆alkyl and halo, or R¹ and R² and/or R³ and R⁴ together with the atoms to which they are attached form a substituted or unsubstituted cycloalkyl; and
M is a transition metal (such as Cr, Mo, Fe, Ru, Os, Co, Rh, Ir, Ni, Pd, Pt, Cu, Ag, and/or Au), optionally bound to another ligand or combination of ligands,
wherein the catalyst-containing monomer does not have the structure 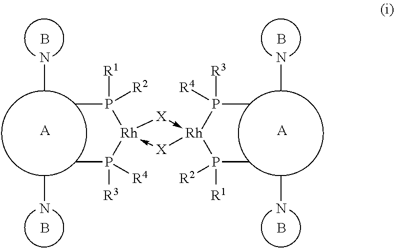
wherein,
R¹, R², R³ and R⁴ are independently selected from phenyl and C₄₋₈cycloalkyl, the latter two groups being unsubstituted or substituted, where possible, with 1, 2, 3, 4, or 5 groups independently selected from C₁₋₆alkyl, OC₁₋₆alkyl and halo, [symbol]
is a binaphthyl group or a derivative of a binaphthyl group, each being unsubstituted or substituted with one or more groups independently selected from C₁₋₆alkyl, OC₁₋₆alkyl and halo,
X is an anionic ligand, and [symbol]
is a monocyclic, bicyclic or tricylic group comprising at least one double bond and being unsubstituted or substituted with one or more groups independently selected from C₁₋₆alkyl, OC₁₋₆alkyl, halo and ═O; 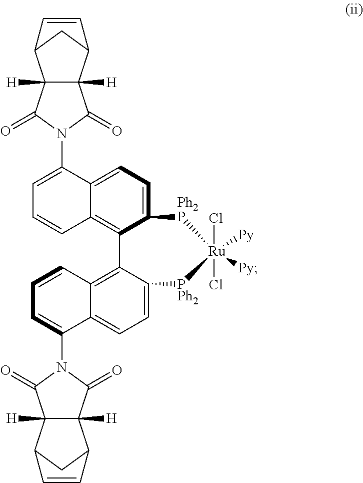
wherein,
Py is an unsubstituted pyridine; or 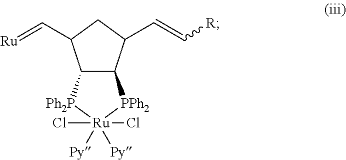
wherein,
R is an unsubstituted phenyl group, and each Py″ is an unsubstituted pyridine ring, or together they are ((1R, 2R)-1,2-diphenylethylenediamine.
